(19) United States
(12) Patent Application Publication
(10) Pub. No.: US 2023/0282363 A1
(43) Pub. Date: Sep. 7, 2023

(54) TECHNIQUES FOR HEALTH-RELATED MINI-INSIGHTS USING WEARABLE DEVICE

(71) Applicant: Oura Health Oy, Oulu (FI)

(72) Inventors: Mari Pauliina Karsikas, Oulu (FI); Johanna Leena Kyllikki Still, Oulu (FI); Henri Matti Johannes Huttunen, Pudasjärvi (FI); Veera Lindholm, Oulu (FI); Petteri Lajunen, Oulu (FI)

(21) Appl. No.: 18/176,986

(22) Filed: Mar. 1, 2023

Related U.S. Application Data
(60) Provisional application No. 63/315,672, filed on Mar. 2, 2022.

(51) Int. Cl. G16H 50/30; G16H 40/63

(52) U.S. Cl. CPC G16H 50/30; G16H 40/63

(57) ABSTRACT

Methods, systems, and devices for providing health-related mini-insights using wearable devices are described. Baseline physiological data is collected from a user via a wearable device, where the baseline physiological data is associated with any physiological parameter, including heart rate data, restorative time, temperature, respiratory rate, blood oxygen saturation, and the like. Additional physiological data is then collected from the user via the wearable device and compared to the user’s baseline physiological data for the respective physiological parameter(s). Based on the comparison, a satisfaction of a trigger condition for providing a health-related insight associated with one or more physiological parameters is identified, and the health-related insight is displayed to the user via a graphical user interface (GUI).

### CROSS REFERENCE

The present Application for Patent claims the benefit of U.S. Provisional Patent Application No. 63/315,672 by KARSIKAS et al., entitled “TECHNIQUES FOR HEALTH-RELATED MINI-INSIGHTS USING A WEARABLE DEVICE,” filed Mar. 2, 2022, assigned to the assignee thereof, and expressly incorporated by reference herein.

### FIELD OF TECHNOLOGY

The following relates to wearable devices and data processing, including techniques for health-related mini-insights using a wearable device.

### BACKGROUND

Some wearable devices may be configured to collect physiological data from users, including temperature data, heart rate data, and the like. Moreover, some wearable devices may provide guidance to the user regarding the user’s acquired physiological data. However, current techniques for providing health-related insights do not provide actionable guidance that enables users to improve their overall health. As such, current techniques for providing health-related insights may be improved.

### BRIEF DESCRIPTION OF THE DRAWINGS

FIG. 1 illustrates an example of a system that supports techniques for health-related mini-insights using a wearable device in accordance with aspects of the present disclosure.

FIG. 2 illustrates an example of a system that supports techniques for health-related mini-insights using a wearable device in accordance with aspects of the present disclosure.

FIG. 3 illustrates an example of a process flow that supports techniques for health-related mini-insights using a wearable device in accordance with aspects of the present disclosure.

FIG. 4 illustrates an example of a process flow that supports techniques for health-related mini-insights using a wearable device in accordance with aspects of the present disclosure.

FIG. 5 illustrates an example of a process flow that supports techniques for health-related mini-insights using a wearable device in accordance with aspects of the present disclosure.

FIG. 6 illustrates an example of a graphical user interface (GUI) that supports techniques for health-related mini-insights using a wearable device in accordance with aspects of the present disclosure.

FIG. 7 illustrates an example of a GUI that supports techniques for health-related mini-insights using a wearable device in accordance with aspects of the present disclosure.

FIG. 8 shows a block diagram of an apparatus that supports techniques for health-related mini-insights using a wearable device in accordance with aspects of the present disclosure.

FIG. 9 shows a block diagram of a wearable application that supports techniques for health-related mini-insights using a wearable device in accordance with aspects of the present disclosure.

FIG. 10 shows a diagram of a system including a device that supports techniques for health-related mini-insights using a wearable device in accordance with aspects of the present disclosure.

FIGS. 11 through 16 show flowcharts illustrating methods that support techniques for health-related mini-insights using a wearable device in accordance with aspects of the present disclosure.

### DETAILED DESCRIPTION

Some wearable devices may be configured to collect physiological data from users, including temperature data, heart rate data, photoplethysmogram (PPG) signals, motion data, and the like. Moreover, some wearable devices may be configured to provide health-related insights to the user regarding the user’s acquired physiological data. However, health-related insights provided by many conventional wearable devices may not enable the users to take action with respect to the health-related insights. That is, many conventional wearable devices often provide guidance to the user at times or cadences that may not enable the user to act upon the provided guidance and improve their overall health. As such, using conventional wearable devices, users may be forced to be reactive with respect to guidance provided by the wearable device, and are unable to proactively take steps to act upon the provided guidance.

Accordingly, aspects of the present disclosure are directed to techniques that enable wearable devices to provide short, actionable guidance throughout the day to enable users to proactively act upon the provided guidance. Stated differently, aspects of the present disclosure are directed to techniques for providing users with “mini-insights” associated with parameters of their overall health to provide the users with more up-to-date and comprehensive depictions of their overall health. By providing users with mini-insights (e.g., “health-related insights”) throughout the day, aspects of the present disclosure may enable users to take action with respect to the mini-insights throughout the day to improve their overall health. That is, as opposed to guidance provided by other wearables which requires users to be reactive with respect to the provided guidance, the “health-related mini-insights” described herein may be triggered at times throughout the day that enable users to take proactive steps to act on the provided guidance and improve their overall health each day.

For example, a wearable device may acquire physiological data from the user throughout the day, such as heart rate data, temperature data, and the like. Physiological parameters used to trigger health-related insights may include any physiological parameters known in the art, including daytime heart rate data (e.g., heart rate while the user is awake), nighttime heart rate data (e.g., heart rate while the user is asleep), restorative time (e.g., time the user spends in a relaxed state), temperature (e.g., body temperature, skin temperature), respiration rate, blood oxygen saturation, activity/movement, blood sugar data (e.g., glucose data), or any combination thereof. Moreover, techniques described herein may be used to provide health-related mini-insights associated with a user’s chronotype to help the user develop habits and routines that are aligned with their personal circadian rhythm.

The wearable device (or another component communicatively coupled to the wearable device) may compare the acquired physiological data to the user’s baseline physiological data to identify “trigger conditions” for providing the user with health-related insights for the current day. Trigger conditions for providing health-related insights (mini-insights) may be satisfied if the user’s physiological data for the current day is higher or lower than the user’s average data for some time period, if the physiological data for the current day is on par with the user’s average, or both. By way of another example, relative timings of naps, bed times, and/or wake times that deviate from the user’s natural circadian rhythm by some threshold time may also be used to trigger chronotype-related mini-insights.

Additionally, or alternatively, a trigger condition for providing a health-related insight may be identified by comparing a “curve” of a physiological parameter associated with the user to a “baseline curve” for the user’s respective physiological parameter. For example, a trigger condition for providing a heart-rate related insight may be identified by comparing a user’s “heart rate curve” (e.g., plot of a user’s heart rate throughout the day or daily trend of the user’s heart rate) to a “baseline heart rate curve” (e.g., average or normal plot of the user’s heart rate throughout a normal day). In such cases, a trigger condition for providing a health-related insight may be based on characteristics of the user’s heart rate curve compared to the baseline heart rate curve, including slope(s) of the curves, shape(s) of the curves, minimum/maximum values of the curves, and the like. Comparison of curves to baseline curves for a user may be used to identify trigger conditions for health-related insights associated with any physiological parameter, including restorative time, temperature (e.g., body temperature, skin temperature), respiration rate, blood oxygen saturation, or any combination thereof.

For example, a system may determine that the user’s daytime heart rate for the current day is higher than the user’s average daytime heart rate over the last week or month by some threshold amount, and may therefore identify a satisfaction of a trigger condition for providing a mini-insight. Additionally, or alternatively, the system may identify a satisfaction of the trigger condition by comparing the user’s heart rate curve (e.g., plot of a user’s heart rate throughout the day or daily trend of the user’s heart rate) to a baseline heart rate curve (e.g., average/typical plot of user’s heart rate throughout the day or average/typical daily trend of the user’s heart rate). In such examples, the system may provide the user with a health-related insight associated with the user’s higher than average daytime heart rate, where the health-related insight may suggest that the user take a moment to relax to help lower their daytime heart rate. As such, by comparing the user’s daytime heart rate to the user’s baseline daytime heart rate, techniques described herein may provide the user with current, up-to-date, and actionable guidance that may enable the user to more efficiently manage and control their heart rate data throughout the day, thereby leading to a healthier lifestyle.

Aspects of the disclosure are initially described in the context of systems supporting physiological data collection from users via wearable devices. Additional aspects of the disclosure are described in the context of example process flows and example graphical user interfaces (GUIs). Aspects of the disclosure are further illustrated by and described with reference to apparatus diagrams, system diagrams, and flowcharts that relate to techniques for health-related mini-insights using a wearable device.

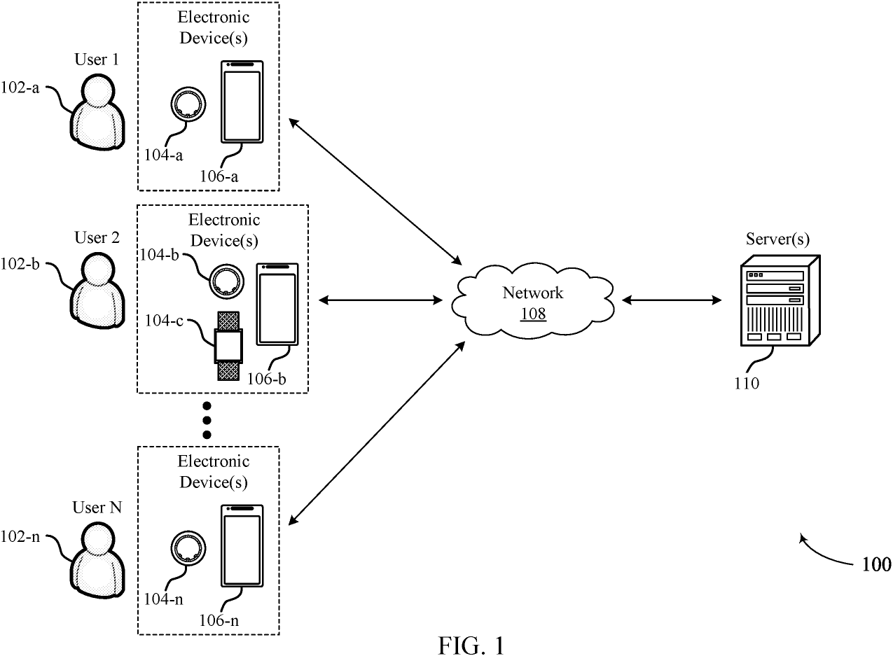

FIG. 1 illustrates an example of a system 100 that supports techniques for health-related mini-insights using a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user’s 102 finger, a wrist computing device (e.g., a smart watch, fitness band, or bracelet) configured to be worn on a user’s 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user’s finger. In particular, a ring 104 may utilize one or more light-emitting components, such as light emitting diodes (LEDs) (e.g., red LEDs, green LEDs) that emit light on the palm-side of a user’s finger to collect physiological data based on arterial blood flow within the user’s finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user’s sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual’s sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user’s natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual’s baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for providing users 102 with health-related insights throughout the day. In other words, the system 100 may support techniques for providing actionable “mini-insights” associated with user’s 102 physiological data to enable users to take action with respect to their physiological data and overall health. The system 200 may collect physiological data from a user 102, determine physiological parameters based on the collected physiological data (e.g., restorative time, daytime heart rate, sleeping heart rate, etc.), and compare the physiological parameters to the user’s baseline data for the respective physiological parameters to identify trigger conditions for providing health-related insights associated with the respective physiological data. Upon identifying a satisfaction of a trigger condition, the system 100 may provide the user with a health-related insight (e.g., mini-insight provided via a GUI of a user device 106). Such health-related insights may provide the user 102 with a more up-to-date and comprehensive view of their health, that enables the user 102 to take action with respect to the health-related insight to improve their overall health.

For example, the system 100 may determine that the user’s daytime heart rate for the current day is higher than the user’s average daytime heart rate over the last week or month by some threshold amount, and may therefore identify a satisfaction of a trigger condition for providing a mini-insight. In this example, the system may provide the user with a health-related insight associated with the user’s higher than average daytime heart rate, where the health-related insight may suggest that the user take a moment to relax to help lower their daytime heart rate. As such, by comparing the user’s daytime heart rate to the user’s baseline daytime heart rate, techniques described herein may provide the user with current, adaptable, up-to-date, and actionable guidance that may enable the user to more efficiently manage and control their heart rate data throughout the day, thereby leading to a healthier lifestyle.

By way of another example, the system 100 may generate a physiological parameter curve/trend for the user for the current sleep day, and may compare the generated physiological parameter curve/trend to a baseline physiological parameter curve/trend to generate mini-insights for the respective physiological parameter. Physiological parameter curves/trends may include graphs, plots, equations, or other data objects that reflect the behavior of the user’s respective physiological parameter (e.g., heart rate, respiratory rate, temperature, blood oxygen saturation, restorative time) over the course of time. Moreover, the baseline physiological parameter curve/trend may be generated based on the user’s own past physiological data, physiological data collected from other users, expected/normal physiological parameter curves/trends, or any combination thereof. In such cases, the system 100 may identify a satisfaction of a trigger condition for a health-related insight based on the comparison of the respective physiological parameter curves to the corresponding baseline physiological parameter curves. In other words, the system 100 may trigger a mini-insight based on one or more parameters of the user’s physiological parameter curve, including a slope, shape, minimum/maximum values, range of values, and the like. Subsequently, the system may provide the user with a health-related insight associated with the user’s physiological parameter (e.g., mini-insights associated with heart rate, respiratory rate, temperature, blood oxygen saturation, restorative time, etc.).

By way of yet another example, the system 100 may determine a quantity of restorative time the user 102 has received throughout the day based on physiological data collected via a wearable device 104. The system 100 may compare the quantity of restorative time for the current day to the user’s baseline (e.g., average restorative time the user has received per day over the last week, month, or other time interval). Based on the comparison, the system 100 may identify a satisfaction of a trigger condition for providing a mini-insight associated with the user’s restorative time, and may display the mini-insight to the user 102 via the user device 106. In this example, the system 100 may determine that the user 102 has received less restorative time compared to the user’s baseline (e.g., satisfy a trigger condition), and the mini-insight may therefore suggest that the user 102 take a minute to relax and recharge.

In some implementations, when comparing a user’s physiological data to the user’s baseline physiological data (e.g., when comparing the user’s daytime heart rate for the current sleep day to the user’s baseline daytime heart rate data), the system 100 may be configured to consider other parameters, conditions, or factors when evaluating whether to trigger a mini-insight. In particular, other parameters and factors associated with the user may be used to shift expectations and/or thresholds associated with the user’s “baseline” data, and to explain “deviations” from the user’s baseline data. Other parameters, conditions, or factors that may be considered when evaluating whether to trigger mini-insights may include, but are not limited to, pregnancy, menstrual cycles, potential illness, excessive/strenuous exercise, vaccinations, and the like.

For example, a user’s temperature, heart rate, and other physiological parameters may naturally change during pregnancy. Simply comparing the user’s physiological data to their “normal” baseline data (e.g., baseline data prior to pregnancy) may result in significant deviations that may thereby trigger concerning insights regarding the user’s physiological data (e.g., a mini-insight that states the user’s temperature is significantly higher than “normal,” despite this being a natural result of pregnancy). Accordingly, techniques described herein may be configured to take the user’s pregnancy (and other conditions/factors) into account when determining whether to trigger mini-insights. For instance, the system 100 may modify the user’s “baseline” data to account for changes in physiological data during pregnancy, create new baseline data for the user during pregnancy, acquire baseline data from other uses that exhibit similar conditions or parameters (e.g., collect baseline data from similar users who are or have been pregnant), and the like. In such cases, the system 100 may compare the user’s physiological data to the adjusted or new baseline data in order to more accurately and reliably determine whether to trigger mini-insights for the user while the user is pregnant.

Moreover, additional conditions/factors may be used to adjust and tailor mini-insights provided to the user. For example, in cases where the system 100 triggers a mini-insights for a user that is currently ovulating or menstruating, the system 100 may tailor the mini-insight to acknowledge or otherwise indicate the user’s ovulation/menstrual cycle as a potential contributing factor for the triggered the mini-insight. For instance, the mini-insight may state that the user’s temperature is on par with the user’s average temperature at similar points in previous ovulation/menstrual cycles, or indicate that the user’s heart rate is higher/lower compared to the user’s average heart rate at similar points in the user’s previous ovulation/menstrual cycles.

When considering external conditions, parameters, and factors to determine whether to trigger mini-insights and/or adjust messaging within mini-insights, the system 100 may be configured to consider subjective data (e.g., “tags”) inputted by the user, as well as data from other applications, such as the user’s calendar (e.g., travel plans). For example, a user may be able to input certain “tags” via the wearable application 250, such as alcohol, caffeine, food, travel, vaccines, stress, meditation, and the like. In such cases, techniques described herein may be configured to take these external tags into account to determine whether the user’s physiological data is departing from the user’s “baseline” due to the conditions associated with the tags, or whether the deviations are attributable to something else. Further, the system 100 may tailor generated mini-insights to acknowledge or otherwise indicate an effect of certain tags on the user’s physiological data. For example, a mini-insight may indicate alcohol as a contributing factor for the mini-insight, or indicate that the user’s heart rate after consuming alcohol is behaving differently compared to how the user’s heart rate typically responds after consuming alcohol in the past.

In some aspects, the system 100 may generate the user’s baseline data for respective physiological parameters based on inputted tags and subjective data (e.g., the user’s respiratory rate typically increases for three hours after consuming caffeine). By taking such subjective data into account, techniques described herein may enable the system 100 to more accurately and efficiently trigger mini-insights that are appropriate for the user and that take the user’s daily routine and habits into account.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

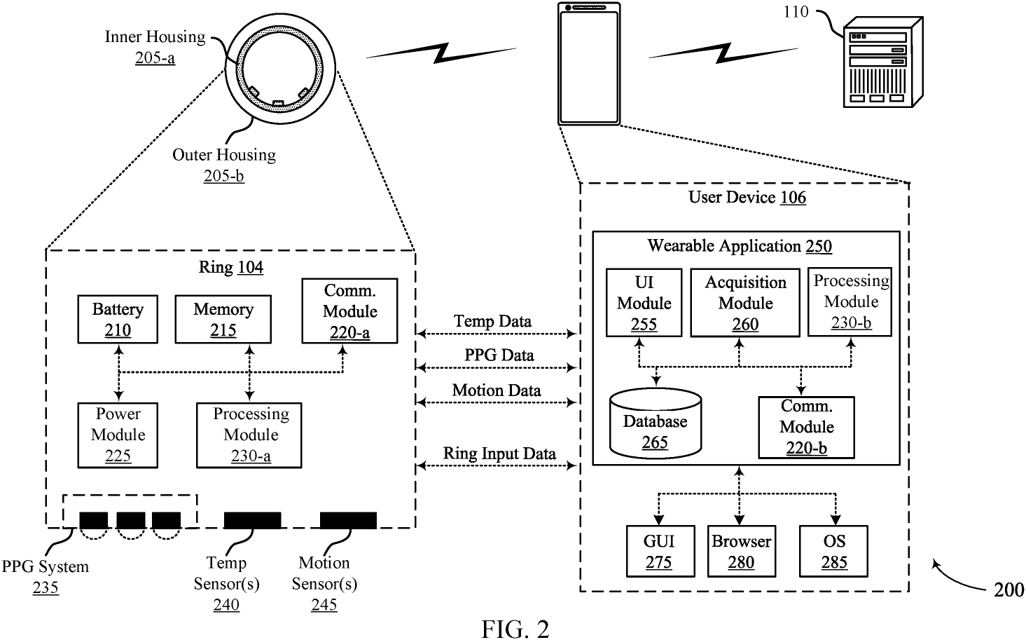

FIG. 2 illustrates an example of a system 200 that supports techniques for health-related mini-insights using a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user’s finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user’s finger. The inner housing 205-a may be formed from a polymer (e.g., a medical grade polymer) or other material. In some implementations, the inner housing 205-a may be transparent. For example, the inner housing 205-a may be transparent to light emitted by the PPG LEDs. In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user’s finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user’s finger (e.g., skin temperature). In some implementations, the temperature sensor 240 may contact the user’s skin. In other implementations, a portion of the housing 205 (e.g., the inner housing 205-a) may form a barrier (e.g., a thin, thermally conductive barrier) between the temperature sensor 240 and the user’s skin. In some implementations, portions of the ring 104 configured to contact the user’s finger may have thermally conductive portions and thermally insulative portions. The thermally conductive portions may conduct heat from the user’s finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user’s temperature over time. For example, the processing module 230-a may sample the user’s temperature according to a sampling rate. An example sampling rate may include one sample per second, although the processing module 230-a may be configured to sample the temperature signal at other sampling rates that are higher or lower than one sample per second. In some implementations, the processing module 230-a may sample the user’s temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user’s finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user’s finger (e.g., any finger). For example, one or more temperature sensors 240 on the ring 104 may acquire a user’s temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 235 may indicate the perfusion of blood in the illuminated region. For example, the PPG signal may indicate blood volume changes in the illuminated region caused by a user’s pulse pressure. The processing module 230-a may sample the PPG signal and determine a user’s pulse waveform based on the PPG signal. The processing module 230-a may determine a variety of physiological parameters based on the user’s pulse waveform, such as a user’s respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user’s finger. In some implementations, the PPG system 235 may be configured as a transmissive PPG system 235 in which the optical transmitter(s) and optical receiver(s) are arranged opposite to one another, such that light is transmitted directly through a portion of the user’s finger to the optical receiver(s).

The number and ratio of transmitters and receivers included in the PPG system 235 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user’s heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user’s respiratory rate over time. For example, the processing module 230-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and Readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user’s finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user’s physiological parameters may be measured by sensors included on a ring 104, other devices may measure a user’s physiological parameters. For example, although a user’s temperature may be measured by a temperature sensor 240 included in a ring 104, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user’s sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5° C. higher than the highest temperature during the longest period. In some aspects, the ring may measure a user’s body temperature while the user is asleep, and the system 200 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for providing users with health-related insights throughout the day. In other words, the system 200 may support techniques for providing actionable “mini-insights” associated with user’s physiological data to enable users to take action with respect to their physiological data and overall health. The system 200 may collect physiological data from a user 102, determine physiological parameters based on the collected physiological data (e.g., restorative time, daytime heart rate, sleeping heart rate, etc.), and compare the physiological parameters to the user’s baseline data for the respective physiological parameters to identify trigger conditions for providing health-related insights associated with the respective physiological data. Upon identifying a satisfaction of a trigger condition, the system 200 may provide the user with a health-related insight (e.g., mini-insight provided via a GUI 275 of a user device 106). Such health-related insights may provide the user 102 with a more up-to-date and comprehensive view of their health, that enables the user 102 to take action with respect to the health-related insight to improve their overall health.

In some aspects, health-related insights (e.g., mini-insights) described herein may be designed to give users contextual and topical highlights and tips on various physiological parameters, including daytime heart rate, heart rate during sleep (e.g., sleeping or nighttime heart rate), and restorative time. Health-related insights may be provided to users via the wearable application 250, as push notifications or other alerts provided via the GUI 275, or any combination thereof. Mini-insights provided throughout the day may improve user engagement with the wearable device 104 and wearable application 250 throughout the day, and may motivate users to take proactive steps to improve their overall health. In this regard, techniques directed to health-related insights (e.g., mini-insights) described herein may help users get more out of their daytime heart rate data, and provide users with highlights and tips that can help the users figure out what to do next, how to schedule their day, and the like. Dynamic insights that keep updating during the day may provide contextual and timely guidance that will help motivate users to visit the wearable application 250 more frequently, and continue to wear the wearable device 104.

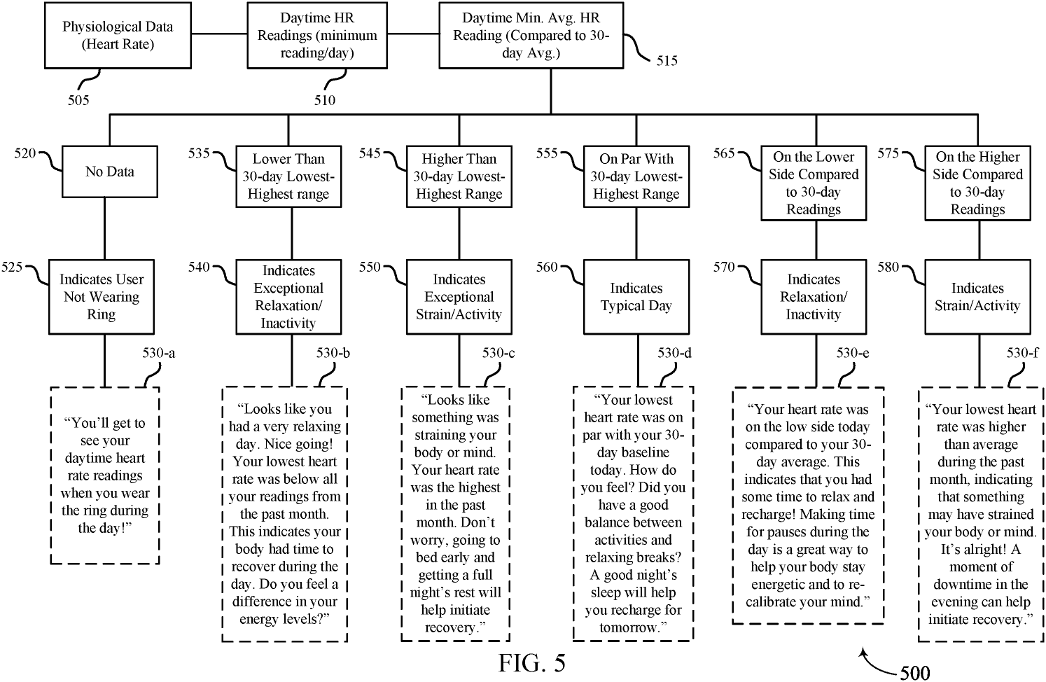

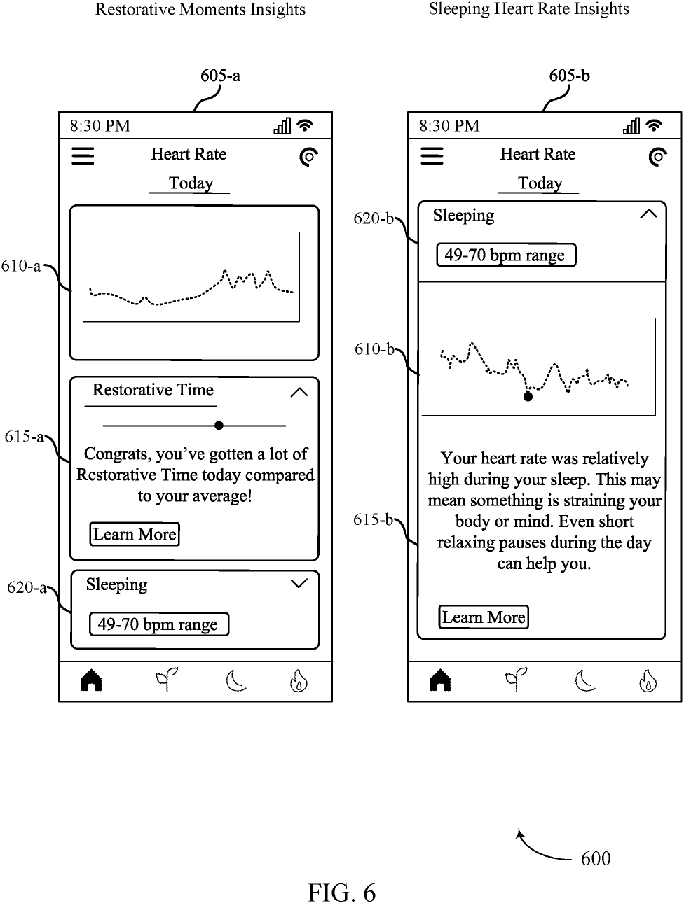

In some aspects, health-related insights may be presented “live” (e.g., in real time or near-real time) via cards presented in the wearable application 250, as will be shown and described in further detail with respect to FIGS. 5 and 6. As it is used herein, the term “health-related insight” and “mini-insight” may be used interchangeably to refer to a message, guidance, or other dynamic content that updates whenever there is new data available for the particular physiological parameter associated with the respective mini-insight. In other words, mini-insights may be updated and provided to users whenever the system 200 identifies a satisfaction of a trigger condition for providing/updating the mini-insight.

In some implementations, trigger conditions for providing/updating a mini-insight may be satisfied if a user’s physiological parameter data is on par with the user’s baseline physiological parameter data, higher/lower than the user’s physiological parameter data by some threshold, any time new data is available, and the like. Additionally, or alternatively, a trigger condition for providing/updating a mini-insight may be satisfied each time the user opens up the wearable application 250 (e.g., the system 200 updates or generates a mini-insight each time the user opens the wearable application 250). Triggering mini-insights each time the user opens the wearable application 250 may ensure the user is provided “fresh” information, thereby increasing user engagement and encouraging the user to take proactive steps to improve their health.

Physiological parameters used to trigger health-related insights may include any physiological parameters known in the art, including daytime heart rate data (e.g., heart rate while the user is awake), nighttime heart rate data (e.g., heart rate while the user is asleep), workout heart rate data (e.g., heart rate during a workout), restorative time (e.g., time the user spends in a relaxed state), temperature, respiration rate, blood oxygen saturation, activity/movement, or any combination thereof.

As noted previously herein, techniques described herein may take into account other conditions, parameters, and factors associated with a user when determining whether to trigger mini-insights. In particular, other parameters and factors associated with the user may be used to shift expectations and/or thresholds associated with the user’s “baseline” data, and to explain “deviations” from the user’s baseline data. Other parameters, conditions, or factors that may be considered when evaluating whether to trigger mini-insights may include, but are not limited to, pregnancy, menstrual cycles, potential illness, excessive/strenuous exercise, vaccinations, and the like. Moreover, messaging and guidance provided within mini-insights may be tailored or modified based on other such conditions and factors.

For example, when evaluating whether to trigger a mini-insight, the system 200 may compare a user’s physiological data to “baseline” physiological data for the user (or other user) collected during periods of similar physiology (e.g., stages of menstrual/ovulation cycle, periods of more or less intense training, periods impacted by environmental factors like weather and hours of daylight, etc.). In other words, the user’s “baseline” data may be adjusted based on other conditions or factors, such as pregnancy, menstrual/ovulation cycles, training programs, daylight savings, seasonal patterns (e.g., less sunlight during winter months), work patterns (e.g., more activity during weekend, less activity during week while at work), and the like.

Moreover, the system 200 may be configured to consider subjective data (e.g., “tags”) inputted by the user, as well as data from other applications, such as the user’s calendar (e.g., travel plans). For example, a user may be able to input certain “tags” via the wearable application 250, such as alcohol, caffeine, food, travel, vaccines, stress, meditation, and the like. In such cases, techniques described herein may be configured to take these external tags into account to determine whether the user’s physiological data is departing from the user’s “baseline” due to the conditions associated with the tags, or whether the deviations are attributable to something else. Further, the system 100 may tailor generated mini-insights to acknowledge or otherwise indicate an effect of certain tags on the user’s physiological data. For example, a mini-insight may indicate alcohol as a contributing factor for the mini-insight, or indicate that the user’s heart rate after consuming alcohol is behaving differently compared to how the user’s heart rate typically responds after consuming alcohol in the past.

In some aspects, the system 200 may generate the user’s baseline data for respective physiological parameters based on inputted tags and subjective data (e.g., the user’s respiratory rate typically increases for three hours after consuming caffeine). By taking such subjective data into account, techniques described herein may enable the system 100 to more accurately and efficiently trigger mini-insights that are appropriate for the user and that take the user’s daily routine and habits into account.

For the purposes of the present disclosure, the terms “restorative moment,” “restorative time,” and like terms, may refer to a time duration when a user is inactive, or otherwise in a relaxed state. For example, in order to efficiently and accurately track a user’s rest patterns, a wearable device may be configured to collect heart rate data and temperature data throughout a 24-hour period, including at night and during the daytime, and may be configured to identify “restorative moments” throughout the day based on the acquired physiological data. In some implementations, the system 200 may be configured to identify restorative time for the user in cases where the user’s heart rate is below a threshold and the user’s temperature is above a threshold (e.g., low heart rate, high temperature).

Health-related insights associated with a user’s restorative time may comment on how much restorative time (e.g., minutes of restorative time) the user has received throughout the day. Mini-insights associated with a user’s restorative time may be triggered or generated whenever there is restorative time identified within daytime heart rate values that are synced, whenever a user creates a “moment” for restorative time, and in cases where the system 200 identifies that the user rested (e.g., meditation, relaxation, etc.). In some cases, “rest” and restorative time may not include time periods that the user is asleep (e.g., a nap may not be considered rest). Mini-insights may also be generated for past days (e.g., previous sleep days) if new data becomes available (e.g., if the wearable device 104 syncs data from a previous sleep day that includes restorative time). Moreover, in some implementations, the system 200 may trigger mini-insights in cases where no restorative time is available (e.g., an absence of restorative time for the current sleep day may be a trigger condition for providing a mini-insight associated with the user’s restorative time).

In this regard, the system 200 may be configured to evaluate and identify multiple trigger conditions for providing mini-insights related to the user’s restorative time for the current sleep day. Trigger conditions for restorative time mini-insights may be satisfied upon syncing new data (e.g., new data including or not including restorative time), an absence of restorative time, and the like. In some cases, the system 200 may evaluate a satisfaction of a trigger condition for triggering mini-insights by comparing the user’s restorative time to the user’s baseline restorative time. For example, the system 200 may compare a quantity of restorative minutes the user has received throughout the current sleep day to the average quantity of restorative minutes the user has received per sleep day over the past week, month, or some other time period.

Trigger conditions for providing restorative time-related mini-insights may be satisfied in multiple circumstances, such as in cases where the system 200 identifies no restorative time, in cases where restorative time is lower than average, in cases where restorative time is higher than average, and in cases where restorative time is on par with average.

If the system 200 identifies no restorative time for the current sleep day and triggers a mini-insight, the mini-insight may encourage the user not to worry about the lack of restorative time, and may encourage the user to reserve time for a pause if that feels needed. If the system 200 identifies that the user’s restorative time for the current sleep day is lower than average and triggers a mini-insight, the mini-insight may congratulate the user on getting some restorative time already, and may encourage the user to make time for pauses if needed. If the system 200 identifies that the user’s restorative time for the current sleep day is higher than average and triggers a mini-insight, the mini-insight may congratulate the user on getting a lot of restorative time. Additionally, or alternatively, if the system 200 identifies that the user’s restorative time for the current sleep day is on par with the user’s average/baseline, the mini-insight may show tips and useful information for the next steps for the day.

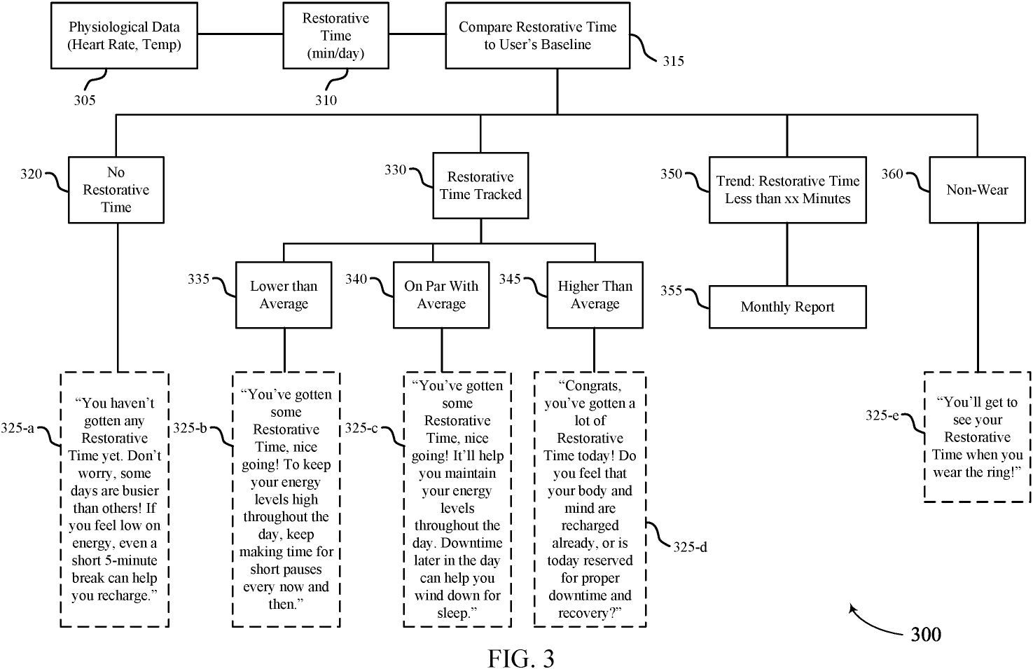

Health-related insights associated with restorative time will be further shown and described with reference to FIG. 3.

Health-related insights associated with a user’s sleeping/nighttime heart rate may comment on the user’s lowest heart rate while the user is sleeping (e.g., lowest sleeping heart rate). Mini-insights associated with sleeping heart rate may be generated if the system 200 detects a long sleep period for the user (and if sleep data is available). Generally, this may occur in the morning after the night’s sleep. In this regard, the system 200 may evaluate and trigger mini-insights associated with sleeping heart rate whenever the longest sleep period for the sleep day occurs. If the system 200 identifies a new longest sleep period during the sleep day, the mini-insight associated with the user’s sleeping heart rate may be updated (e.g., new longest sleep period during sleep day may satisfy trigger condition for mini-insight). For example, if the user takes a nap, at the start of the sleep day (e.g., at 6:00pm), the nap may be the longest sleep period of the sleep day up to that point, and may therefore trigger a sleeping heart rate-related mini-insight. Subsequently, if the user goes to bed at 10:00pm and wakes at 6:00am (the same sleep day), the new sleep period is the new longest sleep period for the sleep day, that may trigger the system 200 to update the mini-insight associated with the user’s sleeping heart rate.

In some implementations, mini-insights associated with the user’s sleeping heart rate may be generated for past days (e.g., past sleep days) if new data becomes available (e.g., if the wearable device 104 syncs new physiological data from past sleep days).

In this regard, the system 200 may be configured to evaluate and identify multiple trigger conditions for providing mini-insights related to the user’s sleeping heart rate for the current sleep day. Trigger conditions for sleeping heart rate mini-insights may be satisfied upon syncing new data (e.g., new longest sleep period, new naps), an absence of sleeping heart rate data, and the like. In some cases, the system 200 may evaluate a satisfaction of a trigger condition for triggering mini-insights by comparing the user’s sleeping heart rate data to the user’s baseline sleeping heart rate data. For example, the system 200 may determine minimum sleeping heart rate values associated with the user for the current sleep day, and may compare the minimum sleeping heart rate values to the user’s baseline sleeping heart rate data (e.g., baseline of lowest sleeping heart rate values) over the past week, month, or some other time period (e.g., resting heart rate contributor assessment).

Trigger conditions for providing mini-insights associated with sleeping heart rate may be satisfied in multiple circumstances, such as in cases where the system 200 identifies no sleeping heart rate data is available, in cases where sleeping heart rate is higher than average, in cases where sleeping heart rate is lower than average, and in cases where sleeping heart rate is on par with average.

Moreover, as described previously herein, mini-insights associated with a user’s heart rate (e.g., sleeping heart rate, daytime heart rate) may be identified by comparing a user’s heart rate curve for the current sleep day to a baseline heart rate curve. The heart rate curves may include graphs, plots, equations, or other data objects that reflect the behavior of the user’s heart rate over the course of time. In such cases, mini-insights may be triggered based on similarities or differences between the user’s heart rate curve and the baseline heart rate curve (e.g., similarities/differences between slopes of curves, shapes of curves, minimum/maximum values of the curves, range of the curves, etc.). Baseline heart rate curves may be generated specifically for the user (e.g., based on the user’s own historical heart rate data), based on physiological data collected from other users (e.g., normal or average heart rate curves across users), and the like.

If the system 200 identifies no sleeping heart rate data for the current sleep day and triggers a mini-insight, such as in cases where the wearable device 104 has low battery or dies during sleep, or in cases with no synced data if the user has not worn the wearable device 104, the mini-insight may show a low-battery message and/or remind the user to wear the wearable device 104. If the system 200 identifies that the user’s sleeping heart rate for the current sleep day is higher than average and triggers a mini-insight (e.g., resting heart rate contributor bar shows “pay attention”), the mini-insight may provide guidance that concentrates on highlighting the importance of recovery and pauses during the day. If the system 200 identifies that the user’s sleeping heart rate for the current sleep day is lower than average and triggers a mini-insight (e.g., resting heart rate contributor bar shows “optimal”), the mini-insight may congratulate the user on great recovery. Additionally, or alternatively, if the system 200 identifies that the user’s sleeping heart rate for the current sleep day is on par with the user’s average/baseline (e.g., resting heart rate contributor bar shows “good”), the mini-insight may provide guidance that concentrates on self-reflection, the user’s daily schedule, and the importance of breaks during the day.

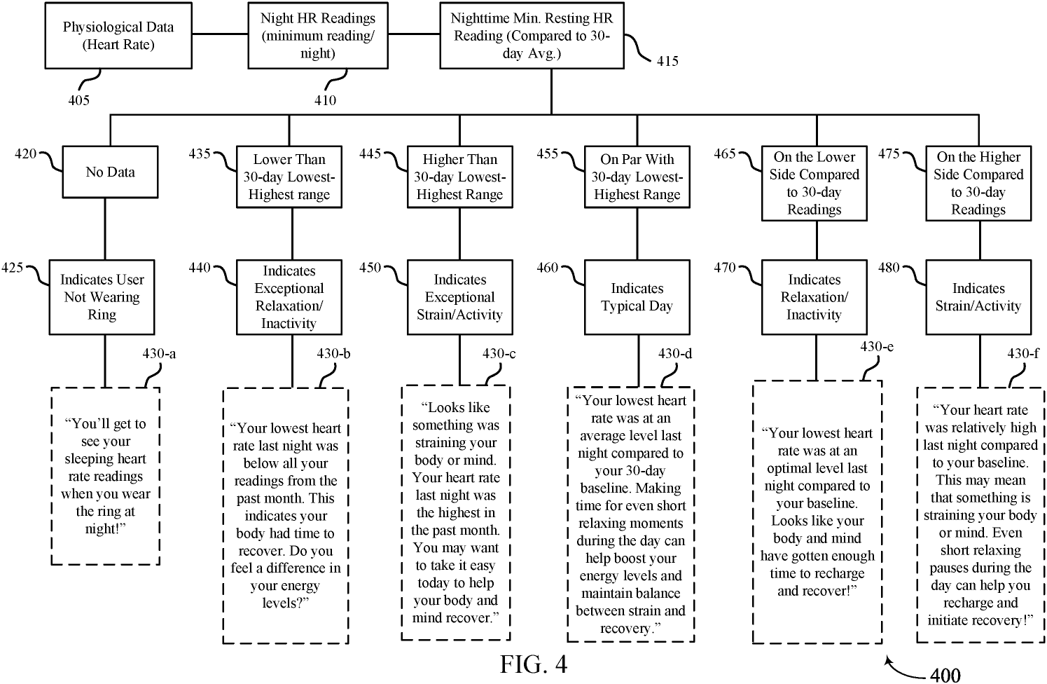

Health-related insights associated with sleeping/nighttime heart rate will be further shown and described with reference to FIG. 4.

Health-related insights associated with a user’s daytime heart rate may comment on the user’s lowest heart rate during the day while the user is awake (e.g., lowest daytime heart rate). Mini-insights associated with daytime heart rate may be generated if the system 200 determines that there is no daytime heart rate available (e.g., if the wearable device 104 has low battery, if the user has not worn the wearable device 104), and in cases where new daytime heart rate data becomes available (e.g., when the wearable device 104 syncs acquired physiological data). In some implementations, mini-insights associated with the user’s daytime heart rate may be generated for past days (e.g., past sleep days) if new data becomes available (e.g., if the wearable device 104 syncs new physiological data from past sleep days).

Mini-insights associated with a user’s daytime heart rate may be generated in three separate phases. During a first phase, the system 200 may collect a lowest average heart rate baseline for the user. For example, the system 200 may collect daytime heart rate data for the user over a time interval (e.g., one week, one month) that will serve as the user’s “baseline” daytime heart rate data. During a second phase, the system 200 may trigger mini-insights associated with the user’s daytime heart rate after sufficient baseline heart rate data has been established (e.g., after collecting baseline heart rate data over 30 days during the first phase 1). Lastly, during a third phase, mini-insights may be further tailored and customized for the user to elaborate on the user’s daytime heart rate and elaborate on the insights.

During the first phase for providing daytime heart rate mini-insights (e.g., before sufficient baseline data is available), the system 200 (e.g., wearable application 250) may provide and display a generic mini-insight throughout the day until some time interval (e.g., two hours) prior to the user’s optimal or baseline bedtime. If the system 200 has not determined a baseline bedtime for the user, the generic mini-insight associated with the user’s daytime heart rate may be displayed until some pre-set time (e.g., 8:00 PM). After the time the generic mini-insight is displayed (e.g., within two hours of the user’s bedtime, or after 8:00 PM), the mini-insight may be changed or updated to a new mini-insight that then shows for the rest of the sleep day.

In this regard, during the first phase, mini-insights during the day may concentrate on guiding the user on how to interpret their heart rate data (e.g., heart rate curve) displayed in the wearable application 250. Comparatively, in the evening, mini-insights may help users find ways to unwind before bedtime. When entering the second phase (e.g., when sufficient baseline data is available), mini-insights associated with daytime heart rate during the day may remain relatively generic, but mini-insights provided in the evenings may comparing the user’s lowest average daytime heart rate for the sleep day to their 30-day lowest average. During the third phase, mini-insights during the day (e.g., before evening summary) may comment on the user’s current lowest average heart rate reading for the current sleep day, and may compare the user’s lowest average heart rate reading to corresponding readings for previous sleep days.

In this regard, the system 200 may be configured to evaluate and identify multiple trigger conditions for providing mini-insights related to the user’s daytime heart rate for the current sleep day. Mini-insights may be evaluated and/or triggered periodically, at irregular or irregular intervals, at specific times (e.g., two hours prior to bedtime, at 8:00 PM), or any combination thereof. Trigger conditions for providing mini-insights associated with daytime heart rate may be satisfied in multiple circumstances, such as in cases where the system 200 identifies no daytime heart rate data is available, in cases where daytime heart rate is much lower than average (e.g., within lowest 5% of the user’s baseline), in cases where daytime heart rate is below average, in cases where daytime heart rate is on par with average, in cases where daytime heart rate is higher than average, and in cases where daytime heart rate is very high (e.g., within 5% of the user’s baseline).

Moreover, as described previously herein, mini-insights associated with a user’s heart rate (e.g., sleeping heart rate, daytime heart rate) may be identified by comparing a user’s heart rate curve for the current sleep day to a baseline heart rate curve. In such cases, mini-insights may be triggered based on similarities or differences between the user’s heart rate curve and the baseline heart rate curve (e.g., similarities/differences between slopes of curves, shapes of curves, minimum/maximum values of the curves, range of the curves, etc.).

If the system 200 identifies no daytime heart rate data for the current sleep day and triggers a mini-insight, such as in cases where there is no synced data or if the user has not worn the wearable device 104, the mini-insight may remind the user to wear the wearable device 104. If the system 200 identifies that the user’s lowest daytime heart rate for the current sleep day is very low (e.g., within lowest 5%) and triggers a mini-insight, the mini-insight may congratulate the user on what seems to have been a restful day. If the system 200 identifies that the user’s lowest daytime heart rate for the current sleep day is lower than average and triggers a mini-insight, the mini-insight may congratulate the user on taking moments of rest and recovery throughout the day.

If the system 200 identifies that the user’s lowest daytime heart rate for the current sleep day is on par with average and triggers a mini-insight, the mini-insight may encourage the user to reflect on their day and schedule (e.g., did the user have all the energy they needed, or would they have benefitted from extra breaks?). If the system 200 identifies that the user’s lowest daytime heart rate for the current sleep day is higher than average and triggers a mini-insight, the mini-insight may encourage the user to reflect on their day and schedule (e.g., how does the user feel after a busy/stressful day like this?) and/or provide tips to unwind for a restful night’s sleep. Lastly, if the system 200 identifies that the user’s lowest daytime heart rate for the current sleep day is on very high (e.g., within highest 5%) and triggers a mini-insight, the mini-insight may emphasize the importance of rest, and provide the user with tips on how to initiate recovery.

Health-related insights associated with daytime heart rate will be further shown and described with reference to FIG. 5.

FIG. 3 illustrates an example of a process flow 300 that supports techniques for health-related mini-insights using a wearable device in accordance with aspects of the present disclosure. Aspects of the process flow 300 may implement, or be implemented by, aspects of the system 100, the system 200, or both. For example, the process flow 300 may be implemented by the system 200 to provide users with mini-insights related to restorative time, as described herein. In this regard, aspects of the process flow 300 may be implemented by one or more components of the system 200, including a wearable device 104, a user device 106, servers 110, or any combination thereof.

At step 305, a wearable device 104 may acquire physiological data from a user. The physiological data may include heart rate data, temperature data, or both. In some cases, the physiological data may include stress-recovery balance data. The stress-recovery balance data may include at least sleep data, the heart rate data, the temperature data, HRV data, and the like.

At 310, the system 200 may determine a quantity of restorative time the user has received. The system 200 may determine a quantity of restorative time the user has received within a given sleep day (e.g., restorative minutes/day). The system 200 may be configured to identify a quantity of restorative time based on the physiological data acquired at step 305. For example, the system 200 may identify that the user has experienced a restorative moment during a time interval, where the user’s heart rate is below some threshold during the time interval, and where the user’s temperature is above some threshold during the time interval.

At step 315, the system 200 may evaluate the identified restorative time, and may compare the restorative time for the current sleep day to the user’s baseline. For example, the system 200 may analyze raw data (e.g., raw restorative time data), analyze a consistency of restorative time between days, and the like. For instance, the system 200 may identify a quantity of restorative moments the user has received within the current sleep day, and may compare the quantity of restorative moments to an average quantity of restorative moments the user has received in the past (e.g., average restorative moments throughout last 30 days). In this regard, the system 200 may compare how much restorative time the user has received compared to their average or baseline over some time interval.

At step 320, the system 200 may identify that the user has not received any restorative time (e.g., zero restorative moments/minutes). In such cases, the process flow 300 may proceed to step 325-a.

At step 325-a, a user device 106 of the system 200 may display a health-related insight (e.g., mini-insight) to the user based on identifying an absence of restorative time at step 320. The health-related insight may read “You haven’t gotten any restorative time yet. Don’t worry, some days are busier than others! If you feel low on energy, even a short 5-minute break can help you recharge.”

In some cases, the mini-insight at step 325-a may be displayed to the user during the day (e.g., during daytime of the respective sleep day). In addition or in the alternate to the mini-insight displayed at step 325-a, the system 200 may display one or more additional health-related insights to the user based on identifying the absence of restorative time at 320. For example, the system 200 may display a health-related insight along with a Readiness message in the morning once the user wakes up during the sleep day, where the health-related insight reads: “You didn’t get any restorative time yesterday. No worries, some days are like that! If you feel tired, how about making time for a short pause now to help your body re-calibrate? Calm inhales and exhales for a couple minutes, or a nice cup of a warm drink of your choice, can work wonders.”

At step 330, the system 200 may identify that the user received some amount of restorative time throughout the respective sleep day. In other words, the system 200 may identify that it has tracked restorative time for the user.

At step 335, the system 200 may identify that the tracked restorative time for the user during the current sleep day is lower than average. For example, the system 200 may identify that the user has received a quantity of restorative moments for the current sleep day, where the quantity of restorative moments is below an average quantity of restorative moments the user has received per day over the last month. Lower than average restorative time may indicate higher levels of stress, strain, activity, or any combination thereof. In such cases, the process flow 300 may proceed to step 325-b.

At step 325-b, a user device 106 of the system 200 may display a health-related insight (e.g., mini-insight) to the user based on identifying that the user’s restorative time for the current sleep day is lower than average at step 335. The health-related insight may read “You’ve gotten some restorative time, nice going! To keep your energy levels high throughout the day, keep making time for short pauses every now and then.”

In some cases, the mini-insight at step 325-b may be displayed to the user during the day (e.g., during daytime of the respective sleep day). In addition or in the alternate to the mini-insight displayed at step 325-b, the system 200 may display one or more additional health-related insights to the user based on identifying that the user’s restorative time for the current sleep day is lower than average at step 335. For example, the system 200 may display a health-related insight along with a Readiness message in the morning once the user wakes up during the sleep day, where the health-related insight reads: “Your restorative time yesterday was a bit on the low side. Don’t worry, even short pauses help you recharge! If you feel like it, how about starting the day with a calm moment of your choice-reading, stretching, breakfast, or something else that makes you feel comfy.”

At step 340, the system 200 may identify that the tracked restorative time for the user during the current sleep day is on par with the user’s average. For example, the system 200 may identify that the user has received an average quantity of restorative moments for the current sleep days as compared to the user’s 30-day baseline. An average amount of restorative time may indicate a normal day for the user with normal balance between strain/activity and relaxation. In such cases, the process flow 300 may proceed to step 325-c.

At step 325-c, a user device 106 of the system 200 may display a health-related insight (e.g., mini-insight) to the user based on identifying an average quantity of restorative time for the current sleep day at step 340. The health-related insight may read “You’ve gotten some restorative time, nice going! It’ll help you maintain your energy levels throughout the day. Downtime later in the day can help you wind down for sleep.”

In some cases, the mini-insight at step 325-c may be displayed to the user during the day (e.g., during daytime of the respective sleep day). In addition or in the alternate to the mini-insight displayed at step 325-c, the system 200 may display one or more additional health-related insights to the user based on identifying that an average quantity of restorative time for the current sleep day at step 340. For example, the system 200 may display a health-related insight along with a Readiness message in the morning once the user wakes up during the sleep day, where the health-related insight reads: “The amount of restorative time you got yesterday was on par with your average. How do you feel, was yesterday’s downtime enough to keep you energetic, or would you want to make time for an additional relaxing moment today?”

At step 345, the system 200 may identify that the tracked restorative time for the user during the current sleep day is higher than average. For example, the system 200 may identify that a quantity of restorative moments the user has received during the current sleep days is greater than the user’s 30-day baseline. Higher than average restorative time may indicate relaxation and inactivity. In such cases, the process flow 300 may proceed to step 325-d.

At step 325-d, a user device 106 of the system 200 may display a health-related insight (e.g., mini-insight) to the user based on identifying a higher than average quantity of restorative time for the current sleep day at 345. The health-related insight may read “Congrats, you’ve gotten a lot of restorative time today! Do you feel that your body and mind are recharged already, or is today reserved for proper downtime and recovery?”

In some cases, the mini-insight at step 325-d may be displayed to the user during the day (e.g., during daytime of the respective sleep day). In addition or in the alternate to the mini-insight displayed at step 325-d, the system 200 may display one or more additional health-related insights to the user based on identifying a higher than average quantity of restorative time for the current sleep day at 345. For example, the system 200 may display a health-related insight along with a Readiness message in the morning once the user wakes up during the sleep day, where the health-related insight reads: “You got more restorative time yesterday than on average, good job! Do you feel the difference? Keep making time for even short pauses during the day, they help refuel your energy levels.”

At 350, the system 200 may identify that the user’s restorative time for the current sleep day is less than a threshold quantity of time (e.g., less than ten minutes). This may indicate strain, stress, activity, and the like. In such cases, the system 200 may include information associated with the restorative time in a monthly report associated with the user at 355. In some cases, the monthly report including the restorative time may be used to address the stress experienced by the user and provide recommendations to the user to handle the stress. For example, the health-related insight may read “You are low on restorative time today and may be experiencing some stress. Take a moment to do five deep inhales and exhales to help cope with the stress you are experiencing.”

At step 360, the system 200 may identify that the user is not (or has not been) wearing the wearable device. For example, in cases where the user is not wearing the wearable device 104, the wearable device may not collect any physiological data at step 305, and the system 200 may determine that the user is not wearing the wearable device based on the absence of retrieved physiological data. In such cases, the process flow 300 may proceed to step 325-e.

At step 325-e, a user device 106 of the system 200 may display a health-related insight (e.g., mini-insight) to the user based on identifying that the user is not wearing (or has not worn) the wearable device at step 360. The health-related insight may read “You’ll get to see your restorative time when you wear the ring!”

In some aspects, the evaluation of the restorative time shown and described throughout the process flow 300 may be performed relative to the quantity of restorative time received at the given point within a sleep day. In particular, the timing of restorative time is important, and restorative time may be evaluated relative to what time it is in the current sleep day. Stated differently, the system 200 may evaluate the user’s restorative time relative to their circadian rhythm.

For example, the system 200 may identify that the user’s restorative time for the current time of day is less than a threshold quantity of restorative time for the user’s circadian rhythm/chronotype. The user device 106 of the system 200 may display a health-related insight (e.g., mini-insight) to the user based on identifying that the user’s restorative time is less than the threshold quantity of restorative time for the user’s circadian rhythm. The health-related insight may read “It looks like you didn’t get as much restorative time for this part of the day that is recommended for your circadian rhythm. Try taking a short pause right now to help refuel your energy levels.”

For example, a user may have received an average of ten restorative minutes per sleep day throughout the past month, and may receive (on average) only two restorative minutes by 11:00 am for each sleep day (where the remaining restorative time is received after 11:00 am for each sleep day). In such cases, if the user has received two restorative minutes by 11:00 am on the current sleep day, they would be “on par” with their average (step 340), and the system 200 may display the mini-insight illustrated at step 325-c. In some cases, the system 200 may display the health-related insights at steps 325-a, 325-b, 325-c, 325-d, and 325-d (and/or additional mini-insights) as push notifications so that the messages are delivered to the user if the user does not open up the wearable application 250.

In other examples, the system 200 may identify that the user’s restorative time for the current time of day is greater than a threshold quantity of restorative time for the user’s circadian rhythm. The user device 106 of the system 200 may display a health-related insight (e.g., mini-insight) to the user based on identifying that the user’s restorative time is greater than the threshold quantity of restorative time for the user’s circadian rhythm. The health-related insight may read “It looks like you got more restorative time for this part of the day based on your circadian rhythm. Congrats! Do you feel your body and mind are refreshed and recharged?”

In some cases, the user device 106 of the system 200 may display a health-related insight (e.g., mini-insight) to the user based on identifying the user’s circadian rhythm. For example, the health-related insight may read “Based on your circadian rhythm, take a moment right now to take a short pause and breathe deeply. The short pause may help refuel your energy levels and unwind your mind.” In such cases, the system 200 may recommend moments for restorative time during the day that align with the user’s circadian rhythm. In some cases, the system 200 may display a health-related insight (e.g., mini-insight) to the user that aligns with the user’s circadian rhythm. For example, the health-related insight may recommend a time of day to nap, rest, meditate, or participate in other activities that contribute to a user’s restorative time.

The process flow 300 shown and described in FIG. 3 may provide users with more frequent and actionable insights regarding their overall health. In particular, the process flow 300 may provide users with more up-to-date messaging that indicates how their restorative time throughout the day may affect their overall health.

In some cases, the system 200 may provide health-related insights to a user based on identifying a circadian rhythm chronotype. For the purposes of the present disclosure, the term “circadian rhythm chronotype” and like terms, may be used to refer to an individual circadian rhythmicity, which is related to sleep, diet, physical activity patterns, and the like. The circadian rhythm is a biological, internal process running in the background of daily functions and orchestrating a twenty-four hour cycle (or an approximately twenty-four hour cycle) in the users. The circadian rhythm regulates biological functions and processes, including but not limited to sleep-wake cycle, alertness, digestion, body temperature, hormone release, and the like. As such, the wearable device 104 may acquire physiological data from a user including at least sleep data, temperature data, activity data, heart rate data, and the like. The temperature data may be an example of continuous nighttime temperature data, and the sleep data may be an example of sleep pattern data or sleep regularity data.

The system 200 may determine the circadian rhythm chronotype. For example, the system 200 may identify that the user is a morning person or an evening person. In such cases, the user device 106 of the system 200 may display a health-related insight (e.g., mini-insight) to the user based on determining the circadian rhythm chronotype. The health-related insight may read “You’re physically active in the morning, you go to bed relatively early, and you wake up early. Your sleep temperature reaches its minimum at almost 2 o′clock,” or “You are a highly active, evening type!” In some cases, the health-related insight may indicate “You are a night wolf” or “You’re an early bird.” The health-related insight may provide insight for the user regarding morning type individuals or evening type individuals. For example, the health-related insight may indicate “Morning types with early bedtimes have a lower risk for cardiovascular disease, and may have lower risks for mental health disorders, including depression, anxiety, and others.”

In some cases, the health-related insight may include a recommended time of day that the user is active, a recommended wake time that the user wakes up, a recommended bedtime that the user goes to sleep, a recommended sleep duration, a recommended time of day that which the user rests, a recommended time that the user is focused, or a combination thereof. In such cases, the system 200 may cause the GUI of the user device to display health-related insights that may provide recommendations to the user based on the determined circadian rhythm chronotype. For example, the health-related insight may read “The optimal time to exercise is 1:30 PM-4:00 PM,” or “6:00 AM-6:30 AM: The sharpest rise in blood pressure, the optimal wake-up time. 7:00 AM-11:00 AM: High alertness, focus on deep or creative work. 2:30 PM-3:00 PM: Afternoon dip. Period of low energy. Take it easy during this time.” In such cases, the health-related insight may include a recommended schedule for the user including bedtimes, wake times, exercise times, focused times, rest times, or a combination thereof. In other examples, the health-related insight may read “You are an evening person, but don’t forget to start winding down for bed at 10:00 PM to help get a good night’s sleep.”

In some cases, the system 200 may evaluate the identified circadian rhythm chronotype, and may compare the circadian rhythm chronotype to the user’s physiological data for the current sleep day. For example, the system 200 may compare the physiological data the system 200 has received within the current sleep day to an average of the physiological data the user has received in the past (e.g., average physiological data throughout last 30 days) that may represent the user’s circadian rhythm chronotype.

In some cases, the system 200 may identify that a circadian rhythm chronotype has not been determined. For example, the system 200 may identify that the absence of physiological data may indicate that the user is not wearing (or has not worn) the wearable device 104 for the current sleep day. In such cases, the user device 106 of the system 200 may display a health-related insight (e.g., mini-insight) to the user based on identifying an absence of a circadian rhythm chronotype. The health-related insight may read “A circadian rhythm chronotype has not been determined for you yet. Don’t worry, you’ll get to see your circadian rhythm chronotype when you wear the ring during the day and night.”

In some examples, the system 200 may identify that the determined circadian rhythm chronotype is misaligned with the received physiological data. For example, the system 200 may identify that the user’s physiological data deviates from the determined circadian rhythm chronotype, such as if a user goes to bed at a time that is out of sync with a recommended bedtime for the user’s circadian rhythm chronotype. In such cases, the user device 106 of the system 200 may display a health-related insight (e.g., mini-insight) to the user based on identifying that the user’s physiological data deviates from the determined circadian rhythm chronotype. The health-related insight may read “Feeling drowsy? Your body is going through a low energy afternoon dip. Don’t worry if you feel lazy; there’s an energy peak coming in an hour,” or “If you feel really low on energy, why not try switching to rest mode for today.”

In some cases, the health-related insight may be an example of an alert generated and displayed to the user via the GUI that may be associated with circadian rhythm chronotype misalignment and recommendations to return to the user’s baseline determined circadian rhythm chronotype. In some cases, the health-related insight may display a recommendation of how to adjust their lifestyle on the day of the determined misalignment and/or in the days after the determined misalignment. The health-related insight may read “Since you went to bed later than usual, devote today for rest,” or “Since you woke up earlier than usual, take some time to rest in the afternoon.”

The system 200 may identify that the circadian rhythm chronotype is aligned with the received physiological data. For example, the system 200 may identify that the user’s physiological data is on par with the determined circadian rhythm chronotype. In such cases, the user device 106 of the system 200 may display a health-related insight (e.g., mini-insight) to the user based on identifying that the user’s physiological data is on par with the determined circadian rhythm chronotype. The health-related insight may read “You are within 85% of your recommended pattern. Keep up the good work,” or “Activity pattern shows you have regular activity in the morning hours, corresponding nicely with your recommended activity window.”

FIG. 4 illustrates an example of a process flow 400 that supports techniques for health-related mini-insights using a wearable device in accordance with aspects of the present disclosure. Aspects of the process flow 400 may implement, or be implemented by, aspects of the system 100, the system 200, or both. For example, the process flow 400 may be implemented by the system 200 to provide users with mini-insights related to sleeping heart rate, as described herein. In this regard, aspects of the process flow 400 may be implemented by one or more components of the system 200, including a wearable device 104, a user device 106, servers 110, or any combination thereof.

At step 405, a wearable device 104 may acquire physiological data from a user. The physiological data may include heart rate data.

At step 410, the system 200 may determine sleeping or nighttime heart rate data for the user. For example, the system 200 may determine minimum heart rate values while the user is sleeping during the current sleep day based on the physiological data acquired at step 405.

At step 415, the system 200 may evaluate the user’s sleeping/nighttime heart rate data, and may compare the sleeping heart rate data for the current sleep day to the user’s baseline sleeping/nighttime heart rate data. For example, the system 200 may compare the user’s minimum sleeping heart rate measurements for the current sleep day to the user’s average range of heart rate values over the past 30 days. In this regard, the system 200 may compare how the user’s sleeping heart rate for the current sleep day compares to the user’s baseline sleeping heart rate over the past month (or some other time interval). In some cases, the system 200 may perform the comparison at step 415 to provide mini-insights regarding the user’s sleeping heart rate in the morning (e.g., once the user wakes up in the moming). In this regard, mini-insights shown and described in process flow 400 may be synced with morning resting heart rate messages. Moreover, the comparison performed at step 415 may be combined with data from the previous sleep day, including total restorative time from the previous sleep day (e.g., restorative time from the previous sleep day may affect the user’s physiological data for the subsequent sleep day).

At step 420, the system 200 may identify that the wearable device 104 has not collected any data related to the user’s sleeping heart rate. In other words, the system 200 may identify that the wearable device 104 did not acquire physiological data at step 405.

At step 425, the system 200 may identify that the absence of physiological data at step 420 may indicate that the user is not wearing (or has not worn) the wearable device 104 for the current sleep day. In such cases, the process flow 400 may proceed to step 430-a.

At step 430-a, a user device 106 of the system 200 may display a health-related insight (e.g., mini-insight) to the user based on identifying an absence of physiological data (e.g., heart rate data) at step 420. The health-related insight may read “You’ll get to see your sleeping heart rate readings when you wear the ring at night!” In some cases, too low of resting/sleeping heart rate values may not necessarily be good. In some cases, low sleeping heart rate values may actually be detrimental to the user’s overall health, and the system 200 may display additional or alternative mini-insights (e.g., “pay attention” mini-insights) associated with the “resting heart rate” contributor.

At step 435, the system 200 may identify that the user’s sleeping minimum heart rate measurements are lower than average. For example, the system 200 may identify that the user’s nighttime minimum resting heart rate is lower than the user’s 30-day lowest-highest range.

At step 440, the system 200 may identify that the lower than average sleeping heart rate identified at step 435 may indicate heightened recovery, inactivity, and/or relaxation as compared to the user’s 30-day baseline. In such cases, the process flow 400 may proceed to step 430-b.

At step 430-b, a user device 106 of the system 200 may display a health-related insight (e.g., mini-insight) to the user based on identifying lower than average sleeping heart rate measurements at step 435. The health-related insight may read “Your lowest heart rate last night was below all your readings from the past month. This indicates your body had time to recover. Do you feel a difference in your energy levels?”

At step 445, the system 200 may identify that the user’s sleeping minimum heart rate measurements are higher than average. For example, the system 200 may identify that the user’s nighttime minimum resting heart rate is higher than the user’s 30-day lowest-highest range.

At step 450, the system 200 may identify that the higher than average sleeping heart rate identified at 445 may indicate heightened strain, activity, etc., as compared to the user’s 30-day baseline. In such cases, the process flow 400 may proceed to step 430-c.

At step 430-c, a user device 106 of the system 200 may display a health-related insight (e.g., mini-insight) to the user based on identifying higher than average sleeping heart rate measurements at step 445. The health-related insight may read “Looks like something was straining your body or mind. Your heart rate last night was the highest in the past month. You may want to take it easy today to help your body and mind recover.”

At step 455, the system 200 may identify that the user’s sleeping minimum heart rate measurements are on par with the user’s average sleeping heart rate measurements. For example, the system 200 may identify that the user’s nighttime minimum resting heart rate is on par with the user’s 30-day lowest-highest range.

At step 460, the system 200 may identify that the average sleeping heart rate identified at step 455 may indicate typical activity/strain and/or typical recovery, inactivity, relaxation, etc., as compared to the user’s 30-day baseline. In other words, the “resting heart rate” contributor for the user for the current sleep day may be considered to be “good,” or normal/average. In such cases, the process flow 400 may proceed to step 430-d.

At step 430-d, a user device 106 of the system 200 may display a health-related insight (e.g., mini-insight) to the user based on identifying average sleeping heart rate measurements at step 455. The health-related insight may read “Your lowest heart rate was at an average level last night compared to your 30-day baseline. Making time for even short relaxing moments during the day can help boost your energy levels and maintain balance between strain and recovery.”

At step 465, the system 200 may identify that the user’s sleeping minimum heart rate measurements are on the lower side compared to the user’s average sleeping heart rate measurements. For example, the system 200 may identify that the user’s nighttime minimum resting heart rate is slightly lower than the user’s 30-day lowest-highest range.

At step 470, the system 200 may identify that the lower than average sleeping heart rate identified at step 465 may indicate increased recovery, inactivity, and/or relaxation as compared to the user’s 30-day baseline. In other words, the “resting heart rate” contributor for the user for the current sleep day may be considered to be “optimal.” In such cases, the process flow 400 may proceed to step 430-e.

At step 430-e, a user device 106 of the system 200 may display a health-related insight (e.g., mini-insight) to the user based on identifying lower than average sleeping heart rate measurements at step 465. The health-related insight may read “Your lowest heart rate was at an optimal level last night compared to your baseline. Looks like your body and mind have gotten enough time to recharge and recover!”

At step 475, the system 200 may identify that the user’s sleeping minimum heart rate measurements are on the higher side compared to the user’s average sleeping heart rate measurements. For example, the system 200 may identify that the user’s nighttime minimum resting heart rate is slightly higher than the user’s 30-day lowest-highest range.

At step 480, the system 200 may identify that the higher than average sleeping heart rate identified at step 475 may indicate increased strain, activity, etc., as compared to the user’s 30-day baseline. In other words, the “resting heart rate” contributor for the user for the current sleep day may be considered to be abnormal, prompting the user to “pay attention” to the resting heart rate contributor. In such cases, the process flow 400 may proceed to step 430-f.

At step 430-f, a user device 106 of the system 200 may display a health-related insight (e.g., mini-insight) to the user based on identifying higher than average sleeping heart rate measurements at step 475. The health-related insight may read “Your heart rate was relatively high last night compared to your baseline. This may mean that something is straining your body or mind. Even short relaxing pauses during the day can help you recharge and initiate recovery!”

The process flow 400 shown and described in FIG. 4 may provide users with more frequent and actionable insights regarding their overall health. In particular, the process flow 400 may provide users with more up-to-date messaging that indicates how their sleeping/nighttime heart rate may affect their overall health.

FIG. 5 illustrates an example of a process flow 500 that supports techniques for health-related mini-insights using a wearable device in accordance with aspects of the present disclosure. Aspects of the process flow 500 may implement, or be implemented by, aspects of the system 100, the system 200, or both. For example, the process flow 500 may be implemented by the system 200 to provide users with mini-insights related to daytime heart rate, as described herein. In this regard, aspects of the process flow 500 may be implemented by one or more components of the system 200, including a wearable device 104, a user device 106, servers 110, or any combination thereof.

At step 505, a wearable device 104 may acquire physiological data from a user. The physiological data may include heart rate data.

At step 510, the system 200 may determine daytime heart rate data for the user. For example, the system 200 may determine minimum heart rate values while the user is awake during the current sleep day based on the physiological data acquired at step 505.

At step 515, the system 200 may evaluate the user’s daytime heart rate data, and may compare the daytime heart rate data for the current sleep day to the user’s baseline daytime heart rate data. For example, the system 200 may compare the user’s minimum daytime heart rate measurements for the current sleep day to the user’s average range of heart rate values over the past 30 days. In this regard, the system 200 may compare how the user’s daytime heart rate for the current sleep day compares to the user’s baseline daytime heart rate over the past month (or some other time interval). In some cases, the system 200 may perform the comparison at 515 to provide mini-insights regarding the user’s daytime heart rate in the evening (e.g., some time interval prior to the user’s typical bedtime, or at some fixed time in the evening).

At step 520, the system 200 may identify that the wearable device 104 has not collected any data related to the user’s daytime heart rate. In other words, the system 200 may identify that the wearable device 104 did not acquire physiological data at step 505.

At step 525, the system 200 may identify that the absence of physiological data at 520 may indicate that the user is not wearing (or has not worn) the wearable device 104 for the current sleep day. In such cases, the process flow 500 may proceed to step 530-a.

At step 530-a, a user device 106 of the system 200 may display a health-related insight (e.g., mini-insight) to the user based on identifying an absence of physiological data (e.g., heart rate data) at 520. The health-related insight may read “You’ll get to see your daytime heart rate readings when you wear the ring during the day!”

At step 535, the system 200 may identify that the user’s daytime heart rate data is lower than average. For example, the system 200 may identify that the user’s daytime minimum resting heart rate is lower than the user’s 30-day lowest-highest range.

At step 540, the system 200 may identify that the lower than average daytime heart rate identified at step 535 may indicate heightened recovery, inactivity, and/or relaxation as compared to the user’s 30-day baseline. In such cases, the process flow 500 may proceed to step 530-b.

At step 530-b, a user device 106 of the system 200 may display a health-related insight (e.g., mini-insight) to the user based on identifying lower than average daytime heart rate measurements at step 535. The health-related insight may read “Looks like you had a very relaxing day. Nice going! Your lowest heart rate was below all your readings from the past month. This indicates your body had time to recover during the day. Do you feel a difference in your energy levels?”

At step 545, the system 200 may identify that the user’s daytime heart rate data is higher than average. For example, the system 200 may identify that the user’s daytime minimum resting heart rate is higher than the user’s 30-day lowest-highest range.

At step 550, the system 200 may identify that the higher than average sleeping heart rate identified at step 545 may indicate heightened strain, activity, etc., as compared to the user’s 30-day baseline. In such cases, the process flow 500 may proceed to step 530-c.

At step 530-c, a user device 106 of the system 200 may display a health-related insight (e.g., mini-insight) to the user based on identifying higher than average sleeping heart rate measurements at step 545. The health-related insight may read “Looks like something was straining your body or mind. Your heart rate was the highest in the past month. Don’t worry, going to bed early and getting a full night’s rest will help initiate recovery.”

At step 555, the system 200 may identify that the user’s daytime heart rate measurements are on par with the user’s average daytime heart rate measurements. For example, the system 200 may identify that the user’s daytime minimum resting heart rate is on par with the user’s 30-day lowest-highest range.

At step 560, the system 200 may identify that the average daytime heart rate identified at step 555 may indicate typical activity/strain and/or typical recovery, inactivity, relaxation, etc., as compared to the user’s 30-day baseline. In such cases, the process flow 500 may proceed to step 530-d.

At step 530-d, a user device 106 of the system 200 may display a health-related insight (e.g., mini-insight) to the user based on identifying average daytime heart rate measurements at step 555. The health-related insight may read “Your lowest heart rate was on par with your 30-day baseline today. How do you feel? Did you have a good balance between activities and relaxing breaks? A good night’s sleep will help you recharge for tomorrow.”

At step 565, the system 200 may identify that the user’s daytime heart rate measurements are on the lower side compared to the user’s daytime sleeping heart rate measurements. For example, the system 200 may identify that the user’s daytime minimum resting heart rate is slightly lower than the user’s 30-day lowest-highest range.

At step 570, the system 200 may identify that the lower than average daytime heart rate identified at step 565 may indicate increased recovery, inactivity, and/or relaxation as compared to the user’s 30-day baseline. In such cases, the process flow 500 may proceed to step 530-e.

At step 530-e, a user device 106 of the system 200 may display a health-related insight (e.g., mini-insight) to the user based on identifying lower than average sleeping heart rate measurements at step 565. The health-related insight may read “Your heart rate was on the low side today compared to your 30-day average. This indicates that you had some time to relax and recharge! Making time for pauses during the day is a great way to help your body stay energetic and to re-calibrate your mind.”

At step 575, the system 200 may identify that the user’s daytime heart rate measurements are on the higher side compared to the user’s average daytime heart rate measurements. For example, the system 200 may identify that the user’s daytime minimum resting heart rate is slightly higher than the user’s 30-day lowest-highest range.

At step 580, the system 200 may identify that the higher than average daytime heart rate identified at step 575 may indicate increased strain, activity, etc., as compared to the user’s 30-day baseline. In such cases, the process flow 500 may proceed to step 530-f.

At step 530-f, a user device 106 of the system 200 may display a health-related insight (e.g., mini-insight) to the user based on identifying higher than average daytime heart rate measurements at step 575. The health-related insight may read “Your lowest heart rate was higher than average during the past month, indicating that something may have strained your body or mind. It’s alright! A moment of downtime in the evening can help initiate recovery.”

The process flow 500 shown and described in FIG. 5 may provide users with more frequent and actionable insights regarding their overall health. In particular, the process flow 500 may provide users with more up-to-date messaging that indicates how their daytime heart rate may affect their overall health.

FIG. 6 illustrates an example of a GUI 600 that supports techniques for health-related mini-insights using a wearable device in accordance with aspects of the present disclosure. The GUI 600 may implement, or be implemented by, aspects of the system 100, system 200, process flow 300, process flow 400, process flow 500, or any combination thereof. For example, the GUI 600 may be an example of a GUI 275 of a user device 106 (e.g., user device 106-a, 106-b, 106-c) corresponding to a user 102.

In some examples, FIG. 6 illustrates a set of application pages 605 that may be displayed to a user 102 via the GUI 600 (e.g., GUI 275 illustrated in FIG. 2). In some implementations, the application pages 605 illustrate details of health-related insights (e.g., mini-insights) that may be provided to a user throughout a sleep day. In particular, the application page 605-a and the application page 605-b illustrate health-related insights associated with restorative time and sleeping heart rate, respectively.

Referring to the first application page 605-a, the GUI 600 may display information associated with a user’s restorative time throughout the current sleep day. For example, the application page 605-a may include a heart rate graph 610-a, a health-related insight 615-a associated with the amount of restorative time the user has received throughout the sleep day, a heart rate card 620-a indicating a sleeping heart rate range for the user during the current sleep day, or any combination thereof.

In some aspects, the GUI 600 may display the first application page 605-a including the health-related insight 615-a associated with the amount of restorative time the user has received throughout the sleep day based on identifying a trigger condition for providing the health-related insight 615-a, as shown and described with reference to FIG. 3. For instance, the GUI 600 may display the health-related insight 615-a associated with the amount of restorative time the user has received throughout the sleep day if the user has received more restorative time than normal, less restorative time than normal, roughly average restorative time, or any combination thereof.

Referring to the second application page 605-b, the GUI 600 may display information associated with a user’s sleeping heart rate throughout the current sleep day. For example, the application page 605-b may include a heart rate graph 610-b, a health-related insight 615-b associated with the user’s sleeping heart rate for the current sleep day, a heart rate card 620-b indicating a sleeping heart rate range for the user during the current sleep day, or any combination thereof.

In some aspects, the GUI 600 may display the second application page 605-b including the health-related insight 615-b associated with the user’s sleeping heart rate based on identifying a trigger condition for providing the health-related insight 615-b, as shown and described with reference to FIG. 4. For instance, the GUI 600 may display the health-related insight 615-b associated with the user’s sleeping heart rate if the user’s lowest sleeping heart rate for the current sleep day is higher than average, lower than average, roughly on par with average, or any combination thereof.

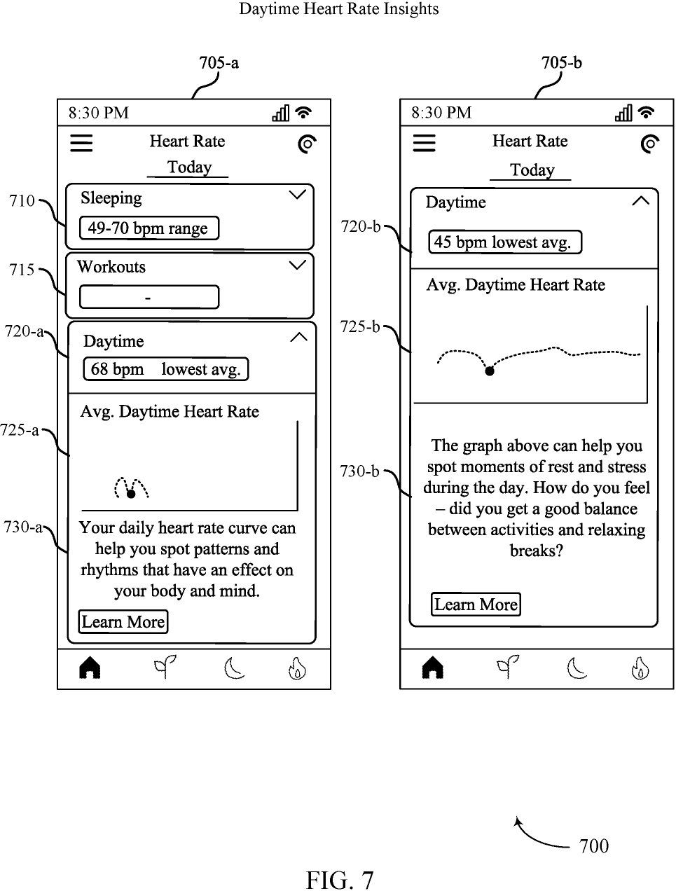

FIG. 7 illustrates an example of a GUI 700 that supports techniques for health-related mini-insights using a wearable device in accordance with aspects of the present disclosure. The GUI 700 may implement, or be implemented by, aspects of the system 100, system 200, process flow 300, process flow 400, process flow 500, or any combination thereof. For example, the GUI 700 may be an example of a GUI 275 of a user device 106 (e.g., user device 106-a, 106-b, 106-c) corresponding to a user 102.

In some examples, FIG. 7 illustrates a set of application pages 705 that may be displayed to a user 102 via the GUI 700 (e.g., GUI 275 illustrated in FIG. 2). In some implementations, the application pages 705 illustrate details of health-related insights (e.g., mini-insights) that may be provided to a user throughout a sleep day. In particular, the application page 705-a and 705-b illustrate health-related insights associated with daytime heart rate.

Referring to the first application page 705-a, the GUI 600 may display information associated with a user’s daytime heart rate throughout the current sleep day. For example, the application page 705-a may include a heart rate card 710 indicating a sleeping heart rate range for the user during the current sleep day, a workouts card 715 indicating workouts the user has completed during the current sleep day, daytime heart rate card 720-a indicating a lowest average daytime heart rate for the current sleep day, a heart rate graph 725-a, a health-related insight 730-a associated with the user’s daytime heart rate for the current sleep day, or any combination thereof. Similarly, referring to the second application page 705-b, the second application page 705-b may include a daytime heart rate card 720-b indicating a lowest average daytime heart rate for the current sleep day, a heart rate graph 725-b, a health-related insight 730-b associated with the user’s daytime heart rate for the current sleep day, or any combination thereof.

In some aspects, the GUI 700 may display the application pages 705-a, 705-b including the health-related insights 730-a, 730-b associated with the user’s daytime heart rate based on identifying a trigger condition for providing the health-related insights 730-a, 730-b, as shown and described with reference to FIG. 5. For instance, the GUI 700 may display the health-related insights 730-a, 730-b associated with the user’s daytime heart rate if the user’s lowest daytime heart rate for the current sleep day is higher than average, lower than average, roughly on par with average, or any combination thereof.

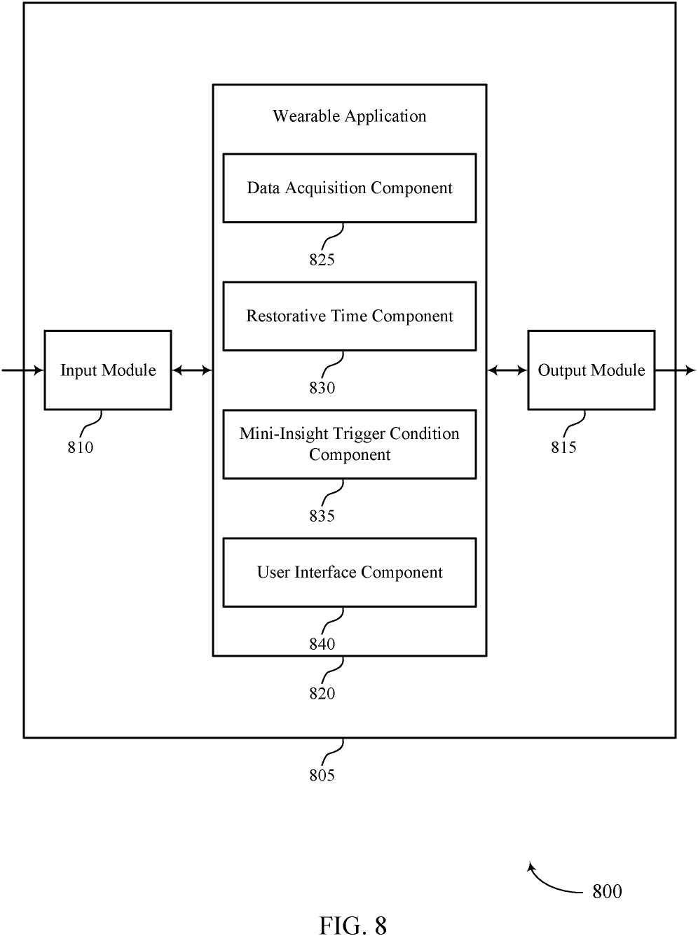

FIG. 8 shows a block diagram 800 of a device 805 that supports techniques for health-related mini-insights using a wearable device in accordance with aspects of the present disclosure. The device 805 may include an input module 810, an output module 815, and a wearable application 820. The device 805 may also include a processor. Each of these components may be in communication with one another (e.g., via one or more buses).

The input module 810 may provide a means for receiving information such as packets, user data, control information, or any combination thereof associated with various information channels (e.g., control channels, data channels, information channels related to illness detection techniques). Information may be passed on to other components of the device 805. The input module 810 may utilize a single antenna or a set of multiple antennas.

The output module 815 may provide a means for transmitting signals generated by other components of the device 805. For example, the output module 815 may transmit information such as packets, user data, control information, or any combination thereof associated with various information channels (e.g., control channels, data channels, information channels related to illness detection techniques). In some examples, the output module 815 may be co-located with the input module 810 in a transceiver module. The output module 815 may utilize a single antenna or a set of multiple antennas.

For example, the wearable application 820 may include a data acquisition component 825, a restorative time component 830, a mini-insight trigger condition component 835, a user interface component 840, or any combination thereof. In some examples, the wearable application 820, or various components thereof, may be configured to perform various operations (e.g., receiving, monitoring, transmitting) using or otherwise in cooperation with the input module 810, the output module 815, or both. For example, the wearable application 820 may receive information from the input module 810, send information to the output module 815, or be integrated in combination with the input module 810, the output module 815, or both to receive information, transmit information, or perform various other operations as described herein.

The wearable application 820 may support providing health-related insights to a user in accordance with examples as disclosed herein. The data acquisition component 825 may be configured as or otherwise support a means for receiving baseline physiological data associated with a user via a wearable device, the baseline physiological data collected throughout a plurality of sleep days, wherein the baseline physiological data comprises at least heart rate data and temperature data. The restorative time component 830 may be configured as or otherwise support a means for identifying baseline restorative moment data associated with the user based at least in part on the heart rate data and the temperature data, the baseline restorative moment data comprising a first quantity of restorative moments within the plurality of sleep days in which the user is in a relaxed state. The data acquisition component 825 may be configured as or otherwise support a means for receiving additional physiological data associated with the user via the wearable device, the additional physiological data collected throughout a first sleep day subsequent to the plurality of sleep days. The restorative time component 830 may be configured as or otherwise support a means for identifying additional restorative moment data associated with the user based at least in part on the additional physiological data, the additional restorative moment data comprising a second quantity of restorative moments within the first sleep day. The mini-insight trigger condition component 835 may be configured as or otherwise support a means for identifying a trigger condition for providing a health-related insight associated with the second quantity of restorative moments based at least in part on a comparison between the additional restorative moment data and the baseline restorative moment data. The user interface component 840 may be configured as or otherwise support a means for causing a graphical user interface to display the health-related insight associated with the additional physiological data.

Additionally, or alternatively, the wearable application 820 may support providing health-related insights to a user in accordance with examples as disclosed herein. The data acquisition component 825 may be configured as or otherwise support a means for receiving baseline heart rate data associated with a user via a wearable device, the baseline heart rate data collected throughout a plurality of sleep days. The data acquisition component 825 may be configured as or otherwise support a means for receiving additional heart rate data associated with the user via the wearable device, the additional heart rate data collected throughout a first sleep day subsequent to the plurality of sleep days. The mini-insight trigger condition component 835 may be configured as or otherwise support a means for identifying a trigger condition for providing a health-related insight associated with the additional heart rate data based at least in part on a comparison between the baseline heart rate data and the additional heart rate data. The user interface component 840 may be configured as or otherwise support a means for causing a graphical user interface to display the health-related insight associated with the additional heart rate data.

Additionally, or alternatively, the wearable application 820 may support providing health-related insights to a user in accordance with examples as disclosed herein. The data acquisition component 825 may be configured as or otherwise support a means for receiving baseline physiological data associated with a user via a wearable device, the baseline physiological data collected throughout a plurality of sleep days, wherein the baseline physiological data comprises at least heart rate data. The data acquisition component 825 may be configured as or otherwise support a means for receiving additional physiological data associated with the user via the wearable device, the additional physiological data collected throughout a first sleep day subsequent to the plurality of sleep days. The mini-insight trigger condition component 835 may be configured as or otherwise support a means for identifying a trigger condition for providing a health-related insight associated with the additional physiological data based at least in part on a comparison between the additional physiological data and the baseline physiological data. The user interface component 840 may be configured as or otherwise support a means for causing a graphical user interface to display the health-related insight associated with the additional physiological data.

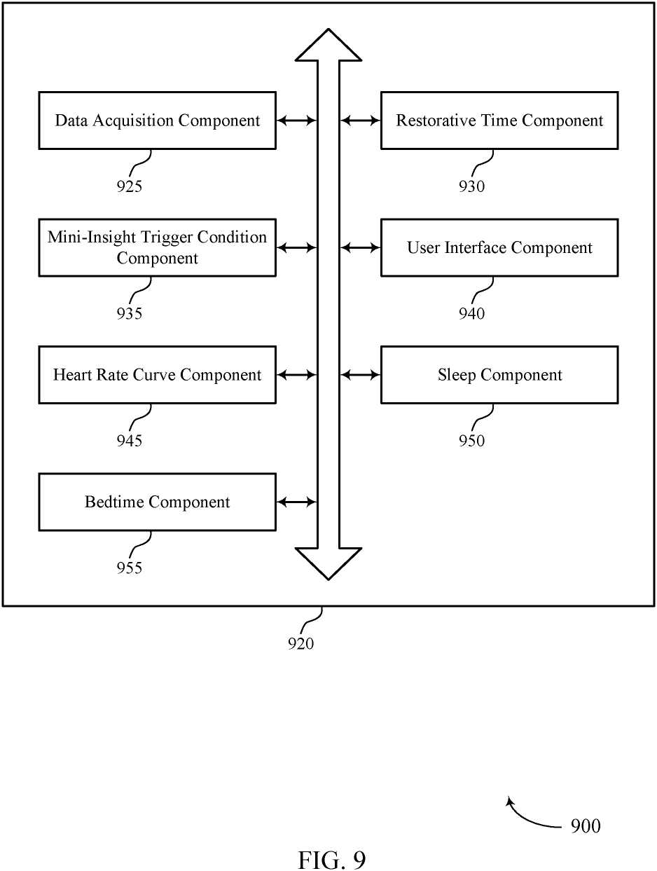

FIG. 9 shows a block diagram 900 of a wearable application 920 that supports techniques for health-related mini-insights using a wearable device in accordance with aspects of the present disclosure. The wearable application 920 may be an example of aspects of a wearable application or a wearable application 820, or both, as described herein. The wearable application 920, or various components thereof, may be an example of means for performing various aspects of techniques for health-related mini-insights using a wearable device as described herein. For example, the wearable application 920 may include a data acquisition component 925, a restorative time component 930, a mini-insight trigger condition component 935, a user interface component 940, a heart rate curve component 945, a sleep component 950, a bedtime component 955, or any combination thereof. Each of these components may communicate, directly or indirectly, with one another (e.g., via one or more buses).

The wearable application 920 may support providing health-related insights to a user in accordance with examples as disclosed herein. The data acquisition component 925 may be configured as or otherwise support a means for receiving baseline physiological data associated with a user via a wearable device, the baseline physiological data collected throughout a plurality of sleep days, wherein the baseline physiological data comprises at least heart rate data and temperature data. The restorative time component 930 may be configured as or otherwise support a means for identifying baseline restorative moment data associated with the user based at least in part on the heart rate data and the temperature data, the baseline restorative moment data comprising a first quantity of restorative moments within the plurality of sleep days in which the user is in a relaxed state. In some examples, the data acquisition component 925 may be configured as or otherwise support a means for receiving additional physiological data associated with the user via the wearable device, the additional physiological data collected throughout a first sleep day subsequent to the plurality of sleep days. In some examples, the restorative time component 930 may be configured as or otherwise support a means for identifying additional restorative moment data associated with the user based at least in part on the additional physiological data, the additional restorative moment data comprising a second quantity of restorative moments within the first sleep day. The mini-insight trigger condition component 935 may be configured as or otherwise support a means for identifying a trigger condition for providing a health-related insight associated with the second quantity of restorative moments based at least in part on a comparison between the additional restorative moment data and the baseline restorative moment data. The user interface component 940 may be configured as or otherwise support a means for causing a graphical user interface to display the health-related insight associated with the additional physiological data.

In some examples, the mini-insight trigger condition component 935 may be configured as or otherwise support a means for identifying the trigger condition based at least in part on a comparison between the first quantity of restorative moments and the second quantity of restorative moments.

In some examples, the baseline physiological data comprises an average quantity of restorative moments per sleep day throughout the plurality of sleep days, and the mini-insight trigger condition component 935 may be configured as or otherwise support a means for identifying the trigger condition based at least in part on a difference between the second quantity of restorative moments and the average quantity of restorative moments satisfying a threshold.

In some examples, wherein the difference satisfies the threshold if the second quantity of restorative moments exceeds the first quantity of restorative moments by a first threshold quantity of restorative moments. In some examples, wherein the difference satisfies the threshold if the second quantity of restorative moments is less than the first quantity of restorative moments by a second threshold quantity of restorative moments.

In some examples, the difference satisfies the threshold if the second quantity of restorative moments is approximately equal to the first quantity of restorative moments.

In some examples, the additional physiological data comprises additional heart rate data and additional temperature data, and the restorative time component 930 may be configured as or otherwise support a means for identifying the first quantity of restorative moments based at least in part on the heart rate data being less than or equal to a heart rate threshold and the temperature data being within a temperature range of a baseline temperature. In some examples, the additional physiological data comprises additional heart rate data and additional temperature data, and the restorative time component 930 may be configured as or otherwise support a means for identifying the second quantity of restorative moments based at least in part on the additional heart rate data being less than or equal to the heart rate threshold and the additional temperature data being within the temperature range of the baseline temperature.

In some examples, the mini-insight trigger condition component 935 may be configured as or otherwise support a means for identifying the trigger condition based at least in part on the second quantity of restorative moments comprising zero restorative moments, wherein the health-related insight is associated with second quantity of restorative moments comprising zero restorative moments.

In some examples, the first quantity of restorative moments and the second quantity of restorative moments comprise time intervals in which the user is awake and in the relaxed state. In some examples, the wearable device comprises a wearable ring device.

Additionally, or alternatively, the wearable application 920 may support providing health-related insights to a user in accordance with examples as disclosed herein. In some examples, the data acquisition component 925 may be configured as or otherwise support a means for receiving baseline heart rate data associated with a user via a wearable device, the baseline heart rate data collected throughout a plurality of sleep days. In some examples, the data acquisition component 925 may be configured as or otherwise support a means for receiving additional heart rate data associated with the user via the wearable device, the additional heart rate data collected throughout a first sleep day subsequent to the plurality of sleep days. In some examples, the mini-insight trigger condition component 935 may be configured as or otherwise support a means for identifying a trigger condition for providing a health-related insight associated with the additional heart rate data based at least in part on a comparison between the baseline heart rate data and the additional heart rate data. In some examples, the user interface component 940 may be configured as or otherwise support a means for causing a graphical user interface to display the health-related insight associated with the additional heart rate data.

In some examples, the baseline heart rate data comprises an first average heart rate per sleep day throughout the plurality of sleep days, and the mini-insight trigger condition component 935 may be configured as or otherwise support a means for identifying the trigger condition based at least in part on a difference between the first average heart rate and the second average heart rate satisfying a threshold.

In some examples, wherein the difference satisfies the threshold if the second average heart rate exceeds the first average heart rate by a first threshold value. In some examples, wherein the difference satisfies the threshold if the second average heart rate is less than the first average heart rate by a second threshold value.

In some examples, the difference satisfies the threshold if the second average heart rate is approximately equal to the first average heart rate.

In some examples, the mini-insight trigger condition component 935 may be configured as or otherwise support a means for identifying the trigger condition based at least in part on the additional heart rate data comprising an indication that the user is not wearing the wearable device, wherein the health-related insight comprises a reminder for the user to wear the wearable device.

In some examples, the heart rate curve component 945 may be configured as or otherwise support a means for generating a baseline heart rate curve associated with the plurality of sleep days based at least in part on the baseline heart rate data. In some examples, the heart rate curve component 945 may be configured as or otherwise support a means for generating a heart rate curve associated with the first sleep day based at least in part on the additional heart rate data, wherein identifying the trigger condition is based at least in part on a comparison of the heart rate curve and the baseline heart rate curve.

In some examples, the heart rate curve component 945 may be configured as or otherwise support a means for generating a baseline heart rate curve based at least in part on a default heart rate curve, physiological data acquired from a plurality of additional users different from the user, or both. In some examples, the heart rate curve component 945 may be configured as or otherwise support a means for generating a heart rate curve associated with the first sleep day based at least in part on the additional heart rate data, wherein identifying the trigger condition is based at least in part on a comparison of the heart rate curve and the baseline heart rate curve.

In some examples, wherein the baseline heart rate data and the additional heart rate data comprise heart rates for the user while the user is awake. In some examples, wherein the baseline heart rate data and the additional heart rate data comprise heart rates for the user while the user is asleep.

In some examples, the sleep component 950 may be configured as or otherwise support a means for identifying the user has awaken from sleep based at least in part on the additional heart rate data, an interaction with the graphical user interface, or both, wherein causing the graphical user interface to display the health-related insight is based at least in part on identifying the user has awaken from sleep.

In some examples, the bedtime component 955 may be configured as or otherwise support a means for determining a bedtime for the user based at least in part on the baseline heart rate data, wherein causing the graphical user interface to display the health-related insight is based at least in part on the bedtime.

In some examples, the baseline heart rate data, the additional heart rate data, or both, are acquired from the user via the wearable device based on arterial blood flow. In some examples, the wearable device comprises a wearable ring device.

Additionally, or alternatively, the wearable application 920 may support providing health-related insights to a user in accordance with examples as disclosed herein. In some examples, the data acquisition component 925 may be configured as or otherwise support a means for receiving baseline physiological data associated with a user via a wearable device, the baseline physiological data collected throughout a plurality of sleep days, wherein the baseline physiological data comprises at least heart rate data. In some examples, the data acquisition component 925 may be configured as or otherwise support a means for receiving additional physiological data associated with the user via the wearable device, the additional physiological data collected throughout a first sleep day subsequent to the plurality of sleep days. In some examples, the mini-insight trigger condition component 935 may be configured as or otherwise support a means for identifying a trigger condition for providing a health-related insight associated with the additional physiological data based at least in part on a comparison between the additional physiological data and the baseline physiological data. In some examples, the user interface component 940 may be configured as or otherwise support a means for causing a graphical user interface to display the health-related insight associated with the additional physiological data.

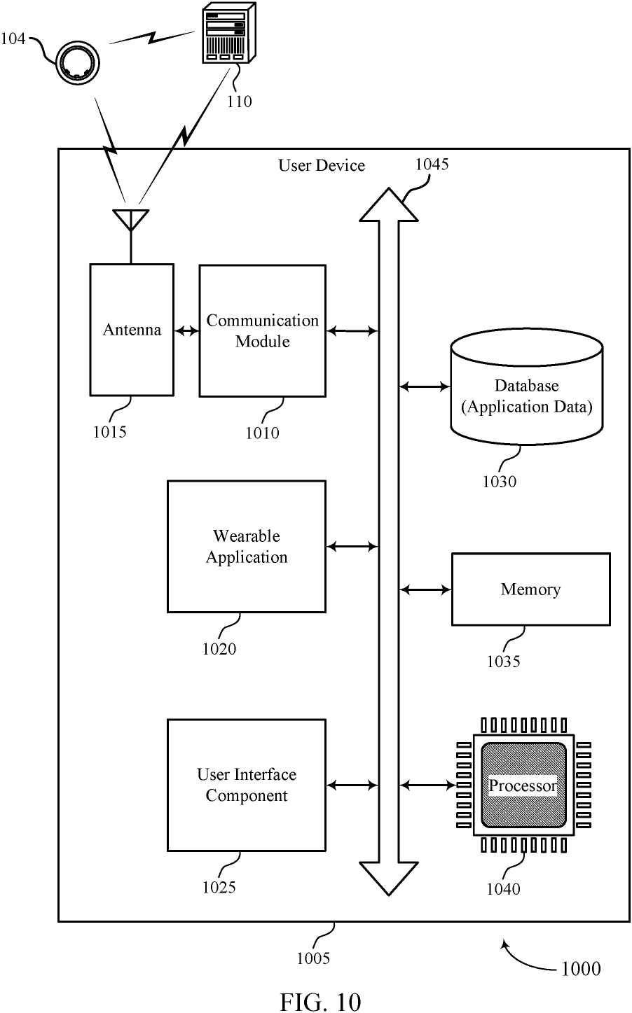

FIG. 10 shows a diagram of a system 1000 including a device 1005 that supports techniques for health-related mini-insights using a wearable device in accordance with aspects of the present disclosure. The device 1005 may be an example of or include the components of a device 805 as described herein. The device 1005 may include an example of a user device 106, as described previously herein. The device 1005 may include components for bi-directional communications including components for transmitting and receiving communications with a wearable device 104 and a server 110, such as a wearable application 1020, a communication module 1010, an antenna 1015, a user interface component 1025, a database (application data) 1030, a memory 1035, and a processor 1040. These components may be in electronic communication or otherwise coupled (e.g., operatively, communicatively, functionally, electronically, electrically) via one or more buses (e.g., a bus 1045).

The communication module 1010 may manage input and output signals for the device 1005 via the antenna 1015. The communication module 1010 may include an example of the communication module 220-b of the user device 106 shown and described in FIG. 2. In this regard, the communication module 1010 may manage communications with the ring 104 and the server 110, as illustrated in FIG. 2. The communication module 1010 may also manage peripherals not integrated into the device 1005. In some cases, the communication module 1010 may represent a physical connection or port to an external peripheral. In some cases, the communication module 1010 may utilize an operating system such as iOS®, ANDROID®, MS-DOS®, MS-WINDOWS®, OS/2®, UNIX®, LINUX®, or another known operating system. In other cases, the communication module 1010 may represent or interact with a wearable device (e.g., ring 104), modem, a keyboard, a mouse, a touchscreen, or a similar device. In some cases, the communication module 1010 may be implemented as part of the processor 1040. In some examples, a user may interact with the device 1005 via the communication module 1010, user interface component 1025, or via hardware components controlled by the communication module 1010.

In some cases, the device 1005 may include a single antenna 1015. However, in some other cases, the device 1005 may have more than one antenna 1015, that may be capable of concurrently transmitting or receiving multiple wireless transmissions. The communication module 1010 may communicate bi-directionally, via the one or more antennas 1015, wired, or wireless links as described herein. For example, the communication module 1010 may represent a wireless transceiver and may communicate bi-directionally with another wireless transceiver. The communication module 1010 may also include a modem to modulate the packets, to provide the modulated packets to one or more antennas 1015 for transmission, and to demodulate packets received from the one or more antennas 1015.

The user interface component 1025 may manage data storage and processing in a database 1030. In some cases, a user may interact with the user interface component 1025. In other cases, the user interface component 1025 may operate automatically without user interaction. The database 1030 may be an example of a single database, a distributed database, multiple distributed databases, a data store, a data lake, or an emergency backup database.

The memory 1035 may include RAM and ROM. The memory 1035 may store computer-readable, computer-executable software including instructions that, when executed, cause the processor 1040 to perform various functions described herein. In some cases, the memory 1035 may contain, among other things, a BIOS that may control basic hardware or software operation such as the interaction with peripheral components or devices.

The processor 1040 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 1040 may be configured to operate a memory array using a memory controller. In other cases, a memory controller may be integrated into the processor 1040. The processor 1040 may be configured to execute computer-readable instructions stored in a memory 1035 to perform various functions (e.g., functions or tasks supporting a method and system for sleep staging algorithms).

The wearable application 1020 may support providing health-related insights to a user in accordance with examples as disclosed herein. For example, the wearable application 1020 may be configured as or otherwise support a means for receiving baseline physiological data associated with a user via a wearable device, the baseline physiological data collected throughout a plurality of sleep days, wherein the baseline physiological data comprises at least heart rate data and temperature data. The wearable application 1020 may be configured as or otherwise support a means for identifying baseline restorative moment data associated with the user based at least in part on the heart rate data and the temperature data, the baseline restorative moment data comprising a first quantity of restorative moments within the plurality of sleep days in which the user is in a relaxed state. The wearable application 1020 may be configured as or otherwise support a means for receiving additional physiological data associated with the user via the wearable device, the additional physiological data collected throughout a first sleep day subsequent to the plurality of sleep days. The wearable application 1020 may be configured as or otherwise support a means for identifying additional restorative moment data associated with the user based at least in part on the additional physiological data, the additional restorative moment data comprising a second quantity of restorative moments within the first sleep day. The wearable application 1020 may be configured as or otherwise support a means for identifying a trigger condition for providing a health-related insight associated with the second quantity of restorative moments based at least in part on a comparison between the additional restorative moment data and the baseline restorative moment data. The wearable application 1020 may be configured as or otherwise support a means for causing a graphical user interface to display the health-related insight associated with the additional physiological data.

Additionally, or alternatively, the wearable application 1020 may support providing health-related insights to a user in accordance with examples as disclosed herein. For example, the wearable application 1020 may be configured as or otherwise support a means for receiving baseline heart rate data associated with a user via a wearable device, the baseline heart rate data collected throughout a plurality of sleep days. The wearable application 1020 may be configured as or otherwise support a means for receiving additional heart rate data associated with the user via the wearable device, the additional heart rate data collected throughout a first sleep day subsequent to the plurality of sleep days. The wearable application 1020 may be configured as or otherwise support a means for identifying a trigger condition for providing a health-related insight associated with the additional heart rate data based at least in part on a comparison between the baseline heart rate data and the additional heart rate data. The wearable application 1020 may be configured as or otherwise support a means for causing a graphical user interface to display the health-related insight associated with the additional heart rate data.

Additionally, or alternatively, the wearable application 1020 may support providing health-related insights to a user in accordance with examples as disclosed herein. For example, the wearable application 1020 may be configured as or otherwise support a means for receiving baseline physiological data associated with a user via a wearable device, the baseline physiological data collected throughout a plurality of sleep days, wherein the baseline physiological data comprises at least heart rate data. The wearable application 1020 may be configured as or otherwise support a means for receiving additional physiological data associated with the user via the wearable device, the additional physiological data collected throughout a first sleep day subsequent to the plurality of sleep days. The wearable application 1020 may be configured as or otherwise support a means for identifying a trigger condition for providing a health-related insight associated with the additional physiological data based at least in part on a comparison between the additional physiological data and the baseline physiological data. The wearable application 1020 may be configured as or otherwise support a means for causing a graphical user interface to display the health-related insight associated with the additional physiological data.

The wearable application 1020 may include an application (e.g., “app”), program, software, or other component that is configured to facilitate communications with a ring 104, server 110, other user devices 106, and the like. For example, the wearable application 1020 may include an application executable on a user device 106 that is configured to receive data (e.g., physiological data) from a ring 104, perform processing operations on the received data, transmit and receive data with the servers 110, and cause presentation of data to a user 102.

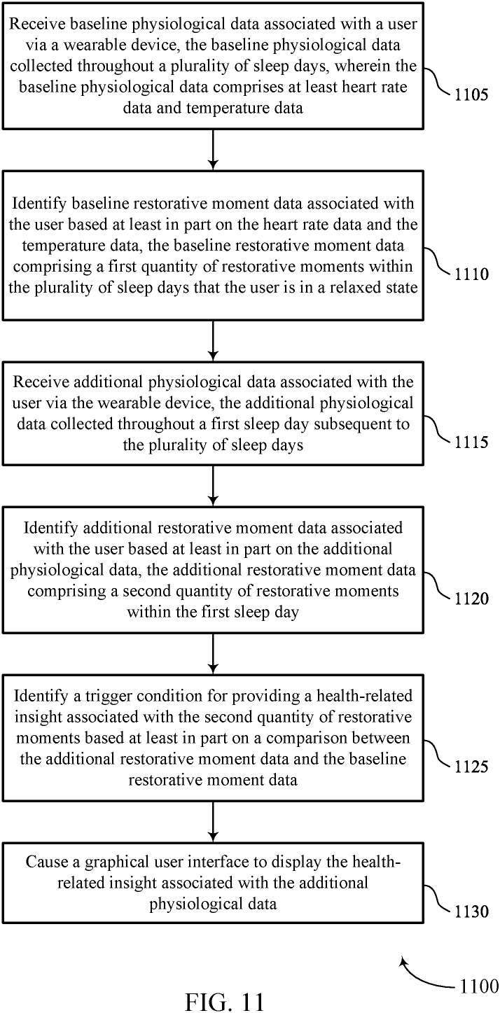

FIG. 11 shows a flowchart illustrating a method 1100 that supports techniques for health-related mini-insights using a wearable device in accordance with aspects of the present disclosure. The operations of the method 1100 may be implemented by a user device or its components as described herein. For example, the operations of the method 1100 may be performed by a user device as described with reference to FIGS. 1 through 10. In some examples, a user device may execute a set of instructions to control the functional elements of the user device to perform the described functions. Additionally, or alternatively, the user device may perform aspects of the described functions using special-purpose hardware.

At 1105, the method may include receiving baseline physiological data associated with a user via a wearable device, the baseline physiological data collected throughout a plurality of sleep days, wherein the baseline physiological data comprises at least heart rate data and temperature data. The operations of 1105 may be performed in accordance with examples as disclosed herein. In some examples, aspects of the operations of 1105 may be performed by a data acquisition component 925 as described with reference to FIG. 9.

At 1110, the method may include identifying baseline restorative moment data associated with the user based at least in part on the heart rate data and the temperature data, the baseline restorative moment data comprising a first quantity of restorative moments within the plurality of sleep days in which the user is in a relaxed state. The operations of 1110 may be performed in accordance with examples as disclosed herein. In some examples, aspects of the operations of 1110 may be performed by a restorative time component 930 as described with reference to FIG. 9.

At 1115, the method may include receiving additional physiological data associated with the user via the wearable device, the additional physiological data collected throughout a first sleep day subsequent to the plurality of sleep days. The operations of 1115 may be performed in accordance with examples as disclosed herein. In some examples, aspects of the operations of 1115 may be performed by a data acquisition component 925 as described with reference to FIG. 9.

At 1120, the method may include identifying additional restorative moment data associated with the user based at least in part on the additional physiological data, the additional restorative moment data comprising a second quantity of restorative moments within the first sleep day. The operations of 1120 may be performed in accordance with examples as disclosed herein. In some examples, aspects of the operations of 1120 may be performed by a restorative time component 930 as described with reference to FIG. 9.

At 1125, the method may include identifying a trigger condition for providing a health-related insight associated with the second quantity of restorative moments based at least in part on a comparison between the additional restorative moment data and the baseline restorative moment data. The operations of 1125 may be performed in accordance with examples as disclosed herein. In some examples, aspects of the operations of 1125 may be performed by a mini-insight trigger condition component 935 as described with reference to FIG. 9.

At 1130, the method may include causing a graphical user interface to display the health-related insight associated with the additional physiological data. The operations of 1130 may be performed in accordance with examples as disclosed herein. In some examples, aspects of the operations of 1130 may be performed by a user interface component 940 as described with reference to FIG. 9.

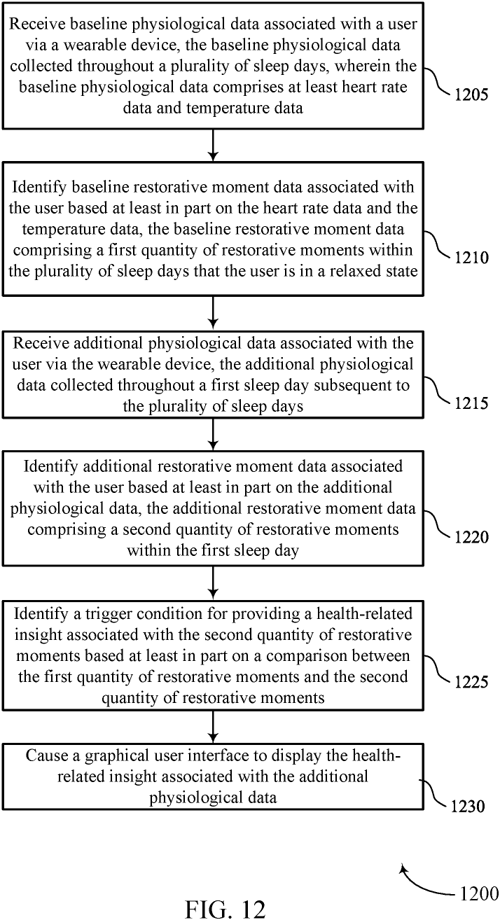

FIG. 12 shows a flowchart illustrating a method 1200 that supports techniques for health-related mini-insights using a wearable device in accordance with aspects of the present disclosure. The operations of the method 1200 may be implemented by a user device or its components as described herein. For example, the operations of the method 1200 may be performed by a user device as described with reference to FIGS. 1 through 10. In some examples, a user device may execute a set of instructions to control the functional elements of the user device to perform the described functions. Additionally, or alternatively, the user device may perform aspects of the described functions using special-purpose hardware.

At 1205, the method may include receiving baseline physiological data associated with a user via a wearable device, the baseline physiological data collected throughout a plurality of sleep days, wherein the baseline physiological data comprises at least heart rate data and temperature data. The operations of 1205 may be performed in accordance with examples as disclosed herein. In some examples, aspects of the operations of 1205 may be performed by a data acquisition component 925 as described with reference to FIG. 9.

At 1210, the method may include identifying baseline restorative moment data associated with the user based at least in part on the heart rate data and the temperature data, the baseline restorative moment data comprising a first quantity of restorative moments within the plurality of sleep days in which the user is in a relaxed state. The operations of 1210 may be performed in accordance with examples as disclosed herein. In some examples, aspects of the operations of 1210 may be performed by a restorative time component 930 as described with reference to FIG. 9.

At 1215, the method may include receiving additional physiological data associated with the user via the wearable device, the additional physiological data collected throughout a first sleep day subsequent to the plurality of sleep days. The operations of 1215 may be performed in accordance with examples as disclosed herein. In some examples, aspects of the operations of 1215 may be performed by a data acquisition component 925 as described with reference to FIG. 9.

At 1220, the method may include identifying additional restorative moment data associated with the user based at least in part on the additional physiological data, the additional restorative moment data comprising a second quantity of restorative moments within the first sleep day. The operations of 1220 may be performed in accordance with examples as disclosed herein. In some examples, aspects of the operations of 1220 may be performed by a restorative time component 930 as described with reference to FIG. 9.

At 1225, the method may include identifying the trigger condition for providing a health-related insight associated with the second quantity of restorative moments based at least in part on a comparison between the first quantity of restorative moments and the second quantity of restorative moments. The operations of 1225 may be performed in accordance with examples as disclosed herein. In some examples, aspects of the operations of 1225 may be performed by a mini-insight trigger condition component 935 as described with reference to FIG. 9.

At 1230, the method may include causing a graphical user interface to display the health-related insight associated with the additional physiological data. The operations of 1230 may be performed in accordance with examples as disclosed herein. In some examples, aspects of the operations of 1230 may be performed by a user interface component 940 as described with reference to FIG. 9.

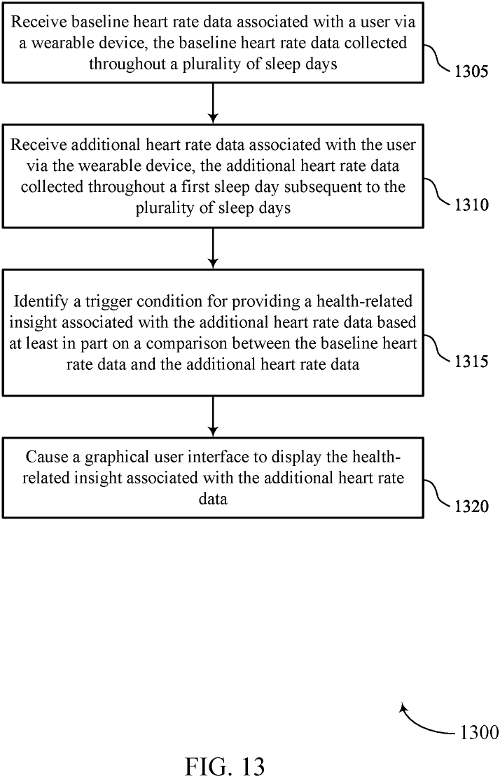

FIG. 13 shows a flowchart illustrating a method 1300 that supports techniques for health-related mini-insights using a wearable device in accordance with aspects of the present disclosure. The operations of the method 1300 may be implemented by a user device or its components as described herein. For example, the operations of the method 1300 may be performed by a user device as described with reference to FIGS. 1 through 10. In some examples, a user device may execute a set of instructions to control the functional elements of the user device to perform the described functions. Additionally, or alternatively, the user device may perform aspects of the described functions using special-purpose hardware.

At 1305, the method may include receiving baseline heart rate data associated with a user via a wearable device, the baseline heart rate data collected throughout a plurality of sleep days. The operations of 1305 may be performed in accordance with examples as disclosed herein. In some examples, aspects of the operations of 1305 may be performed by a data acquisition component 925 as described with reference to FIG. 9.

At 1310, the method may include receiving additional heart rate data associated with the user via the wearable device, the additional heart rate data collected throughout a first sleep day subsequent to the plurality of sleep days. The operations of 1310 may be performed in accordance with examples as disclosed herein. In some examples, aspects of the operations of 1310 may be performed by a data acquisition component 925 as described with reference to FIG. 9.

At 1315, the method may include identifying a trigger condition for providing a health-related insight associated with the additional heart rate data based at least in part on a comparison between the baseline heart rate data and the additional heart rate data. The operations of 1315 may be performed in accordance with examples as disclosed herein. In some examples, aspects of the operations of 1315 may be performed by a mini-insight trigger condition component 935 as described with reference to FIG. 9.

At 1320, the method may include causing a graphical user interface to display the health-related insight associated with the additional heart rate data. The operations of 1320 may be performed in accordance with examples as disclosed herein. In some examples, aspects of the operations of 1320 may be performed by a user interface component 940 as described with reference to FIG. 9.

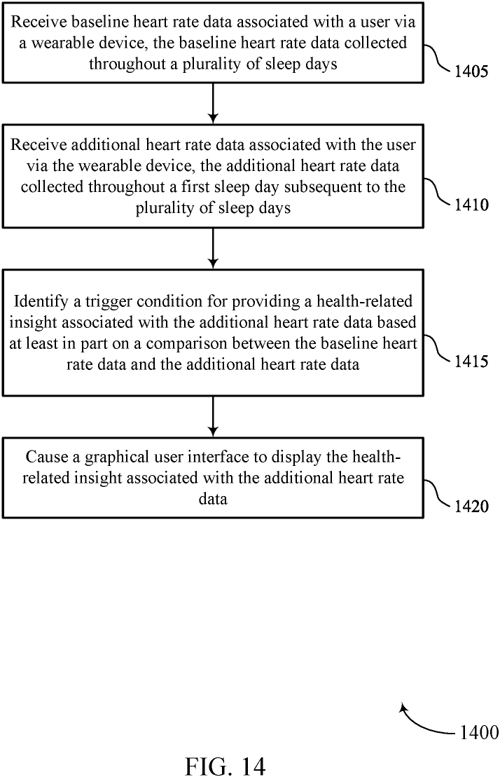

FIG. 14 shows a flowchart illustrating a method 1400 that supports techniques for health-related mini-insights using a wearable device in accordance with aspects of the present disclosure. The operations of the method 1400 may be implemented by a user device or its components as described herein. For example, the operations of the method 1400 may be performed by a user device as described with reference to FIGS. 1 through 10. In some examples, a user device may execute a set of instructions to control the functional elements of the user device to perform the described functions. Additionally, or alternatively, the user device may perform aspects of the described functions using special-purpose hardware.

At 1405, the method may include receiving baseline heart rate data associated with a user via a wearable device, the baseline heart rate data collected throughout a plurality of sleep days. The operations of 1405 may be performed in accordance with examples as disclosed herein. In some examples, aspects of the operations of 1405 may be performed by a data acquisition component 925 as described with reference to FIG. 9.

At 1410, the method may include receiving additional heart rate data associated with the user via the wearable device, the additional heart rate data collected throughout a first sleep day subsequent to the plurality of sleep days. The operations of 1410 may be performed in accordance with examples as disclosed herein. In some examples, aspects of the operations of 1410 may be performed by a data acquisition component 925 as described with reference to FIG. 9.

At 1415, the method may include identifying a trigger condition for providing a health-related insight associated with the additional heart rate data based at least in part on a comparison between the baseline heart rate data and the additional heart rate data. The operations of 1415 may be performed in accordance with examples as disclosed herein. In some examples, aspects of the operations of 1415 may be performed by a mini-insight trigger condition component 935 as described with reference to FIG. 9.

At 1420, the method may include causing a graphical user interface to display the health-related insight associated with the additional heart rate data. The operations of 1420 may be performed in accordance with examples as disclosed herein. In some examples, aspects of the operations of 1420 may be performed by a user interface component 940 as described with reference to FIG. 9.

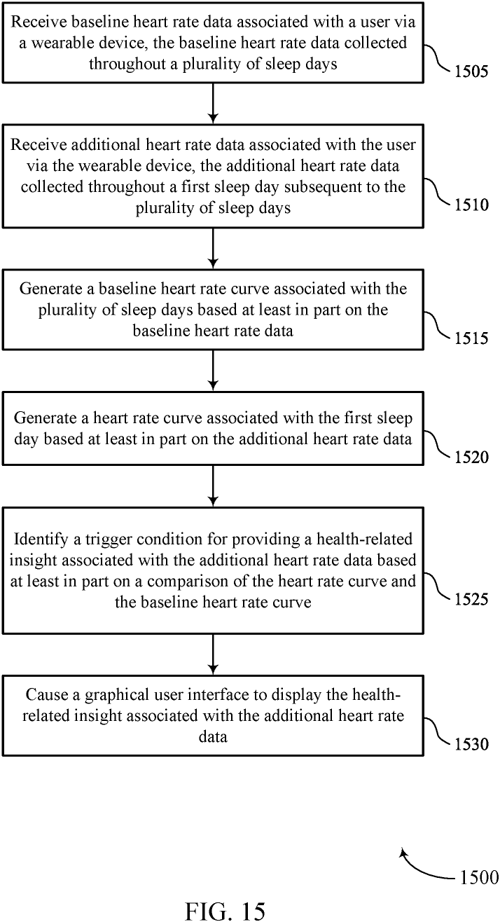

FIG. 15 shows a flowchart illustrating a method 1500 that supports techniques for health-related mini-insights using a wearable device in accordance with aspects of the present disclosure. The operations of the method 1500 may be implemented by a user device or its components as described herein. For example, the operations of the method 1500 may be performed by a user device as described with reference to FIGS. 1 through 10. In some examples, a user device may execute a set of instructions to control the functional elements of the user device to perform the described functions. Additionally, or alternatively, the user device may perform aspects of the described functions using special-purpose hardware.

At 1505, the method may include receiving baseline heart rate data associated with a user via a wearable device, the baseline heart rate data collected throughout a plurality of sleep days. The operations of 1505 may be performed in accordance with examples as disclosed herein. In some examples, aspects of the operations of 1505 may be performed by a data acquisition component 925 as described with reference to FIG. 9.

At 1510, the method may include receiving additional heart rate data associated with the user via the wearable device, the additional heart rate data collected throughout a first sleep day subsequent to the plurality of sleep days. The operations of 1510 may be performed in accordance with examples as disclosed herein. In some examples, aspects of the operations of 1510 may be performed by a data acquisition component 925 as described with reference to FIG. 9.

At 1515, the method may include generating a baseline heart rate curve associated with the plurality of sleep days based at least in part on the baseline heart rate data. The operations of 1515 may be performed in accordance with examples as disclosed herein. In some examples, aspects of the operations of 1515 may be performed by a heart rate curve component 945 as described with reference to FIG. 9.

At 1520, the method may include generating a heart rate curve associated with the first sleep day based at least in part on the additional heart rate data. The operations of 1520 may be performed in accordance with examples as disclosed herein. In some examples, aspects of the operations of 1520 may be performed by a heart rate curve component 945 as described with reference to FIG. 9.

At 1525, the method may include identifying a trigger condition for providing a health-related insight associated with the additional heart rate data based at least in part on a comparison of the heart rate curve and the baseline heart rate curve. The operations of 1525 may be performed in accordance with examples as disclosed herein. In some examples, aspects of the operations of 1525 may be performed by a mini-insight trigger condition component 935 as described with reference to FIG. 9.

At 1530, the method may include causing a graphical user interface to display the health-related insight associated with the additional heart rate data. The operations of 1530 may be performed in accordance with examples as disclosed herein. In some examples, aspects of the operations of 1530 may be performed by a user interface component 940 as described with reference to FIG. 9.

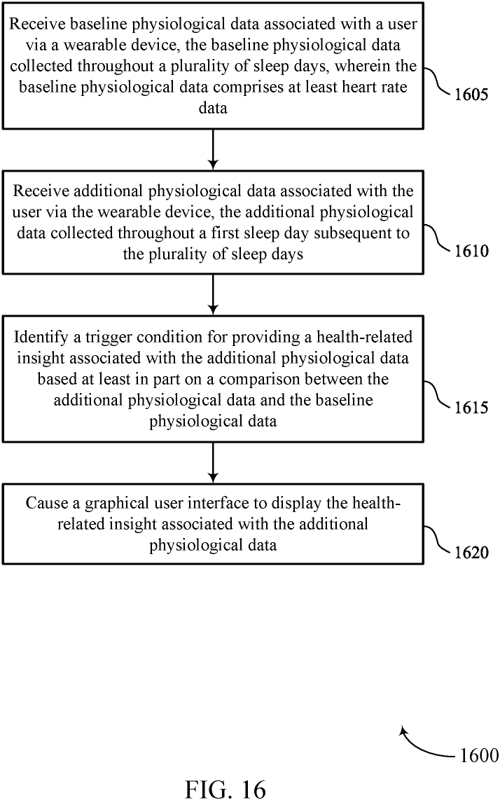

FIG. 16 shows a flowchart illustrating a method 1600 that supports techniques for health-related mini-insights using a wearable device in accordance with aspects of the present disclosure. The operations of the method 1600 may be implemented by a user device or its components as described herein. For example, the operations of the method 1600 may be performed by a user device as described with reference to FIGS. 1 through 10. In some examples, a user device may execute a set of instructions to control the functional elements of the user device to perform the described functions. Additionally, or alternatively, the user device may perform aspects of the described functions using special-purpose hardware.

At 1605, the method may include receiving baseline physiological data associated with a user via a wearable device, the baseline physiological data collected throughout a plurality of sleep days, wherein the baseline physiological data comprises at least heart rate data. The operations of 1605 may be performed in accordance with examples as disclosed herein. In some examples, aspects of the operations of 1605 may be performed by a data acquisition component 925 as described with reference to FIG. 9.

At 1610, the method may include receiving additional physiological data associated with the user via the wearable device, the additional physiological data collected throughout a first sleep day subsequent to the plurality of sleep days. The operations of 1610 may be performed in accordance with examples as disclosed herein. In some examples, aspects of the operations of 1610 may be performed by a data acquisition component 925 as described with reference to FIG. 9.

At 1615, the method may include identifying a trigger condition for providing a health-related insight associated with the additional physiological data based at least in part on a comparison between the additional physiological data and the baseline physiological data. The operations of 1615 may be performed in accordance with examples as disclosed herein. In some examples, aspects of the operations of 1615 may be performed by a mini-insight trigger condition component 935 as described with reference to FIG. 9.

At 1620, the method may include causing a graphical user interface to display the health-related insight associated with the additional physiological data. The operations of 1620 may be performed in accordance with examples as disclosed herein. In some examples, aspects of the operations of 1620 may be performed by a user interface component 940 as described with reference to FIG. 9.

It should be noted that the methods described above describe possible implementations, and that the operations and the steps may be rearranged or otherwise modified and that other implementations are possible. Furthermore, aspects from two or more of the methods may be combined.

A method for providing health-related insights to a user is described. The method may include receiving baseline physiological data associated with a user via a wearable device, the baseline physiological data collected throughout a plurality of sleep days, wherein the baseline physiological data comprises at least heart rate data and temperature data, identifying baseline restorative moment data associated with the user based at least in part on the heart rate data and the temperature data, the baseline restorative moment data comprising a first quantity of restorative moments within the plurality of sleep days in which the user is in a relaxed state, receiving additional physiological data associated with the user via the wearable device, the additional physiological data collected throughout a first sleep day subsequent to the plurality of sleep days, identifying additional restorative moment data associated with the user based at least in part on the additional physiological data, the additional restorative moment data comprising a second quantity of restorative moments within the first sleep day, identifying a trigger condition for providing a health-related insight associated with the second quantity of restorative moments based at least in part on a comparison between the additional restorative moment data and the baseline restorative moment data, and causing a graphical user interface to display the health-related insight associated with the additional physiological data.

An apparatus for providing health-related insights to a user is described. The apparatus may include a processor, memory coupled with the processor, and instructions stored in the memory. The instructions may be executable by the processor to cause the apparatus to receive baseline physiological data associated with a user via a wearable device, the baseline physiological data collected throughout a plurality of sleep days, wherein the baseline physiological data comprises at least heart rate data and temperature data, identify baseline restorative moment data associated with the user based at least in part on the heart rate data and the temperature data, the baseline restorative moment data comprising a first quantity of restorative moments within the plurality of sleep days in which the user is in a relaxed state, receive additional physiological data associated with the user via the wearable device, the additional physiological data collected throughout a first sleep day subsequent to the plurality of sleep days, identify additional restorative moment data associated with the user based at least in part on the additional physiological data, the additional restorative moment data comprising a second quantity of restorative moments within the first sleep day, identify a trigger condition for providing a health-related insight associated with the second quantity of restorative moments based at least in part on a comparison between the additional restorative moment data and the baseline restorative moment data, and cause a graphical user interface to display the health-related insight associated with the additional physiological data.

Another apparatus for providing health-related insights to a user is described. The apparatus may include means for receiving baseline physiological data associated with a user via a wearable device, the baseline physiological data collected throughout a plurality of sleep days, wherein the baseline physiological data comprises at least heart rate data and temperature data, means for identifying baseline restorative moment data associated with the user based at least in part on the heart rate data and the temperature data, the baseline restorative moment data comprising a first quantity of restorative moments within the plurality of sleep days in which the user is in a relaxed state, means for receiving additional physiological data associated with the user via the wearable device, the additional physiological data collected throughout a first sleep day subsequent to the plurality of sleep days, means for identifying additional restorative moment data associated with the user based at least in part on the additional physiological data, the additional restorative moment data comprising a second quantity of restorative moments within the first sleep day, means for identifying a trigger condition for providing a health-related insight associated with the second quantity of restorative moments based at least in part on a comparison between the additional restorative moment data and the baseline restorative moment data, and means for causing a graphical user interface to display the health-related insight associated with the additional physiological data.

A non-transitory computer-readable medium storing code for providing health-related insights to a user is described. The code may include instructions executable by a processor to receive baseline physiological data associated with a user via a wearable device, the baseline physiological data collected throughout a plurality of sleep days, wherein the baseline physiological data comprises at least heart rate data and temperature data, identify baseline restorative moment data associated with the user based at least in part on the heart rate data and the temperature data, the baseline restorative moment data comprising a first quantity of restorative moments within the plurality of sleep days in which the user is in a relaxed state, receive additional physiological data associated with the user via the wearable device, the additional physiological data collected throughout a first sleep day subsequent to the plurality of sleep days, identify additional restorative moment data associated with the user based at least in part on the additional physiological data, the additional restorative moment data comprising a second quantity of restorative moments within the first sleep day, identify a trigger condition for providing a health-related insight associated with the second quantity of restorative moments based at least in part on a comparison between the additional restorative moment data and the baseline restorative moment data, and cause a graphical user interface to display the health-related insight associated with the additional physiological data.

Some examples of the method, apparatuses, and non-transitory computer-readable medium described herein may further include operations, features, means, or instructions for identifying the trigger condition based at least in part on a comparison between the first quantity of restorative moments and the second quantity of restorative moments.

In some examples of the method, apparatuses, and non-transitory computer-readable medium described herein, the baseline physiological data comprises an average quantity of restorative moments per sleep day throughout the plurality of sleep days and the method, apparatuses, and non-transitory computer-readable medium may include further operations, features, means, or instructions for identifying the trigger condition based at least in part on a difference between the second quantity of restorative moments and the average quantity of restorative moments satisfying a threshold.

In some examples of the method, apparatuses, and non-transitory computer-readable medium described herein, wherein the difference satisfies the threshold if the second quantity of restorative moments exceeds the first quantity of restorative moments by a first threshold quantity of restorative moments and wherein the difference satisfies the threshold if the second quantity of restorative moments may be less than the first quantity of restorative moments by a second threshold quantity of restorative moments.

In some examples of the method, apparatuses, and non-transitory computer-readable medium described herein, the difference satisfies the threshold if the second quantity of restorative moments may be approximately equal to the first quantity of restorative moments.

In some examples of the method, apparatuses, and non-transitory computer-readable medium described herein, the additional physiological data comprises additional heart rate data and additional temperature data and the method, apparatuses, and non-transitory computer-readable medium may include further operations, features, means, or instructions for identifying the first quantity of restorative moments based at least in part on the heart rate data being less than or equal to a heart rate threshold and the temperature data being within a temperature range of a baseline temperature and identifying the second quantity of restorative moments based at least in part on the additional heart rate data being less than or equal to the heart rate threshold and the additional temperature data being within the temperature range of the baseline temperature.

Some examples of the method, apparatuses, and non-transitory computer-readable medium described herein may further include operations, features, means, or instructions for identifying the trigger condition based at least in part on the second quantity of restorative moments comprising zero restorative moments, wherein the health-related insight may be associated with second quantity of restorative moments comprising zero restorative moments.

In some examples of the method, apparatuses, and non-transitory computer-readable medium described herein, the first quantity of restorative moments and the second quantity of restorative moments comprise time intervals in which the user may be awake and in the relaxed state.

In some examples of the method, apparatuses, and non-transitory computer-readable medium described herein, the wearable device comprises a wearable ring device.

A method for providing health-related insights to a user is described. The method may include receiving baseline heart rate data associated with a user via a wearable device, the baseline heart rate data collected throughout a plurality of sleep days, receiving additional heart rate data associated with the user via the wearable device, the additional heart rate data collected throughout a first sleep day subsequent to the plurality of sleep days, identifying a trigger condition for providing a health-related insight associated with the additional heart rate data based at least in part on a comparison between the baseline heart rate data and the additional heart rate data, and causing a graphical user interface to display the health-related insight associated with the additional heart rate data.

An apparatus for providing health-related insights to a user is described. The apparatus may include a processor, memory coupled with the processor, and instructions stored in the memory. The instructions may be executable by the processor to cause the apparatus to receive baseline heart rate data associated with a user via a wearable device, the baseline heart rate data collected throughout a plurality of sleep days, receive additional heart rate data associated with the user via the wearable device, the additional heart rate data collected throughout a first sleep day subsequent to the plurality of sleep days, identify a trigger condition for providing a health-related insight associated with the additional heart rate data based at least in part on a comparison between the baseline heart rate data and the additional heart rate data, and cause a graphical user interface to display the health-related insight associated with the additional heart rate data.

Another apparatus for providing health-related insights to a user is described. The apparatus may include means for receiving baseline heart rate data associated with a user via a wearable device, the baseline heart rate data collected throughout a plurality of sleep days, means for receiving additional heart rate data associated with the user via the wearable device, the additional heart rate data collected throughout a first sleep day subsequent to the plurality of sleep days, means for identifying a trigger condition for providing a health-related insight associated with the additional heart rate data based at least in part on a comparison between the baseline heart rate data and the additional heart rate data, and means for causing a graphical user interface to display the health-related insight associated with the additional heart rate data.

A non-transitory computer-readable medium storing code for providing health-related insights to a user is described. The code may include instructions executable by a processor to receive baseline heart rate data associated with a user via a wearable device, the baseline heart rate data collected throughout a plurality of sleep days, receive additional heart rate data associated with the user via the wearable device, the additional heart rate data collected throughout a first sleep day subsequent to the plurality of sleep days, identify a trigger condition for providing a health-related insight associated with the additional heart rate data based at least in part on a comparison between the baseline heart rate data and the additional heart rate data, and cause a graphical user interface to display the health-related insight associated with the additional heart rate data.

In some examples of the method, apparatuses, and non-transitory computer-readable medium described herein, the baseline heart rate data comprises an first average heart rate per sleep day throughout the plurality of sleep days and the method, apparatuses, and non-transitory computer-readable medium may include further operations, features, means, or instructions for identifying the trigger condition based at least in part on a difference between the first average heart rate and the second average heart rate satisfying a threshold.

In some examples of the method, apparatuses, and non-transitory computer-readable medium described herein, wherein the difference satisfies the threshold if the second average heart rate exceeds the first average heart rate by a first threshold value and wherein the difference satisfies the threshold if the second average heart rate may be less than the first average heart rate by a second threshold value.

In some examples of the method, apparatuses, and non-transitory computer-readable medium described herein, the difference satisfies the threshold if the second average heart rate may be approximately equal to the first average heart rate.

Some examples of the method, apparatuses, and non-transitory computer-readable medium described herein may further include operations, features, means, or instructions for identifying the trigger condition based at least in part on the additional heart rate data comprising an indication that the user may be not wearing the wearable device, wherein the health-related insight comprises a reminder for the user to wear the wearable device.

Some examples of the method, apparatuses, and non-transitory computer-readable medium described herein may further include operations, features, means, or instructions for generating a baseline heart rate curve associated with the plurality of sleep days based at least in part on the baseline heart rate data and generating a heart rate curve associated with the first sleep day based at least in part on the additional heart rate data, wherein identifying the trigger condition may be based at least in part on a comparison of the heart rate curve and the baseline heart rate curve.

Some examples of the method, apparatuses, and non-transitory computer-readable medium described herein may further include operations, features, means, or instructions for generating a baseline heart rate curve based at least in part on a default heart rate curve, physiological data acquired from a plurality of additional users different from the user, or both and generating a heart rate curve associated with the first sleep day based at least in part on the additional heart rate data, wherein identifying the trigger condition may be based at least in part on a comparison of the heart rate curve and the baseline heart rate curve.

In some examples of the method, apparatuses, and non-transitory computer-readable medium described herein, wherein the baseline heart rate data and the additional heart rate data comprise heart rates for the user while the user may be awake and wherein the baseline heart rate data and the additional heart rate data comprise heart rates for the user while the user may be asleep.

Some examples of the method, apparatuses, and non-transitory computer-readable medium described herein may further include operations, features, means, or instructions for identifying the user may have awaken from sleep based at least in part on the additional heart rate data, an interaction with the graphical user interface, or both, wherein causing the graphical user interface to display the health-related insight may be based at least in part on identifying the user may have awaken from sleep.

Some examples of the method, apparatuses, and non-transitory computer-readable medium described herein may further include operations, features, means, or instructions for determining a bedtime for the user based at least in part on the baseline heart rate data, wherein causing the graphical user interface to display the health-related insight may be based at least in part on the bedtime.

In some examples of the method, apparatuses, and non-transitory computer-readable medium described herein, the baseline heart rate data, the additional heart rate data, or both, may be acquired from the user via the wearable device based on arterial blood flow.

In some examples of the method, apparatuses, and non-transitory computer-readable medium described herein, the wearable device comprises a wearable ring device.

A method for providing health-related insights to a user is described. The method may include receiving baseline physiological data associated with a user via a wearable device, the baseline physiological data collected throughout a plurality of sleep days, wherein the baseline physiological data comprises at least heart rate data, receiving additional physiological data associated with the user via the wearable device, the additional physiological data collected throughout a first sleep day subsequent to the plurality of sleep days, identifying a trigger condition for providing a health-related insight associated with the additional physiological data based at least in part on a comparison between the additional physiological data and the baseline physiological data, and causing a graphical user interface to display the health-related insight associated with the additional physiological data.

An apparatus for providing health-related insights to a user is described. The apparatus may include a processor, memory coupled with the processor, and instructions stored in the memory. The instructions may be executable by the processor to cause the apparatus to receive baseline physiological data associated with a user via a wearable device, the baseline physiological data collected throughout a plurality of sleep days, wherein the baseline physiological data comprises at least heart rate data, receive additional physiological data associated with the user via the wearable device, the additional physiological data collected throughout a first sleep day subsequent to the plurality of sleep days, identify a trigger condition for providing a health-related insight associated with the additional physiological data based at least in part on a comparison between the additional physiological data and the baseline physiological data, and cause a graphical user interface to display the health-related insight associated with the additional physiological data.

Another apparatus for providing health-related insights to a user is described. The apparatus may include means for receiving baseline physiological data associated with a user via a wearable device, the baseline physiological data collected throughout a plurality of sleep days, wherein the baseline physiological data comprises at least heart rate data, means for receiving additional physiological data associated with the user via the wearable device, the additional physiological data collected throughout a first sleep day subsequent to the plurality of sleep days, means for identifying a trigger condition for providing a health-related insight associated with the additional physiological data based at least in part on a comparison between the additional physiological data and the baseline physiological data, and means for causing a graphical user interface to display the health-related insight associated with the additional physiological data.

A non-transitory computer-readable medium storing code for providing health-related insights to a user is described. The code may include instructions executable by a processor to receive baseline physiological data associated with a user via a wearable device, the baseline physiological data collected throughout a plurality of sleep days, wherein the baseline physiological data comprises at least heart rate data, receive additional physiological data associated with the user via the wearable device, the additional physiological data collected throughout a first sleep day subsequent to the plurality of sleep days, identify a trigger condition for providing a health-related insight associated with the additional physiological data based at least in part on a comparison between the additional physiological data and the baseline physiological data, and cause a graphical user interface to display the health-related insight associated with the additional physiological data.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for providing health-related insights to a user, comprising:
receiving baseline physiological data associated with a user via a wearable device, the baseline physiological data collected throughout a plurality of sleep days, wherein the baseline physiological data comprises at least heart rate data;
receiving additional physiological data associated with the user via the wearable device, the additional physiological data collected throughout a first sleep day subsequent to the plurality of sleep days;
identifying a trigger condition for providing a health-related insight associated with the additional physiological data based at least in part on a comparison between the additional physiological data and the baseline physiological data; and
causing a graphical user interface to display the health-related insight associated with the additional physiological data.

2. The method of claim 1, further comprising:
identifying a circadian rhythm chronotype associated with the user based at least in part on the baseline physiological data, wherein identifying the trigger condition is based at least in part on the circadian rhythm chronotype.

3. The method of claim 1, wherein the baseline physiological data further comprises temperature data, the method further comprising:
identifying baseline restorative moment data associated with the user based at least in part on the heart rate data and the temperature data, the baseline restorative moment data comprising a first quantity of restorative moments within the plurality of sleep days in which the user is in a relaxed state; and
identifying additional restorative moment data associated with the user based at least in part on the additional physiological data, the additional restorative moment data comprising a second quantity of restorative moments within the first sleep day, wherein identifying the trigger condition for providing the health-related insight is based at least in part on a comparison between the baseline restorative moment data and the baseline restorative moment data.

4. The method of claim 3, further comprising:
identifying the trigger condition based at least in part on a comparison between the first quantity of restorative moments and the second quantity of restorative moments.

5. The method of claim 3, wherein the baseline physiological data comprises an average quantity of restorative moments per sleep day throughout the plurality of sleep days, the method further comprising:
identifying the trigger condition based at least in part on a difference between the second quantity of restorative moments and the average quantity of restorative moments satisfying a threshold.

6. The method of claim 5,
wherein the difference satisfies the threshold if the second quantity of restorative moments exceeds the first quantity of restorative moments by a first threshold quantity of restorative moments, or
wherein the difference satisfies the threshold if the second quantity of restorative moments is less than the first quantity of restorative moments by a second threshold quantity of restorative moments.

7. The method of claim 3, wherein the additional physiological data comprises additional heart rate data and additional temperature data, the method further comprising:
identifying the first quantity of restorative moments based at least in part on the heart rate data being less than or equal to a heart rate threshold and the temperature data being within a temperature range of a baseline temperature; and
identifying the second quantity of restorative moments based at least in part on the additional heart rate data being less than or equal to the heart rate threshold and the additional temperature data being within the temperature range of the baseline temperature.

8. The method of claim 3, wherein the first quantity of restorative moments and the second quantity of restorative moments comprise time intervals in which the user is awake and in the relaxed state.

9. The method of claim 1, wherein the wearable device comprises a wearable ring device.

10. A method for providing health-related insights to a user, comprising:
receiving baseline heart rate data associated with a user via a wearable device, the baseline heart rate data collected throughout a plurality of sleep days;
receiving additional heart rate data associated with the user via the wearable device, the additional heart rate data collected throughout a first sleep day subsequent to the plurality of sleep days;
identifying a trigger condition for providing a health-related insight associated with the additional heart rate data based at least in part on a comparison between the baseline heart rate data and the additional heart rate data; and
causing a graphical user interface to display the health-related insight associated with the additional heart rate data.

11. The method of claim 10, wherein the baseline heart rate data comprises an first average heart rate per sleep day throughout the plurality of sleep days, and wherein the additional heart rate data comprises a second average heart rate throughout the first sleep day, the method further comprising:
identifying the trigger condition based at least in part on a difference between the first average heart rate and the second average heart rate satisfying a threshold.

12. The method of claim 11,
wherein the difference satisfies the threshold if the second average heart rate exceeds the first average heart rate by a first threshold value, or
wherein the difference satisfies the threshold if the second average heart rate is less than the first average heart rate by a second threshold value.

13. The method of claim 11, wherein the difference satisfies the threshold if the second average heart rate is approximately equal to the first average heart rate.

14. The method of claim 10, further comprising:
identifying the trigger condition based at least in part on the additional heart rate data comprising an indication that the user is not wearing the wearable device, wherein the health-related insight comprises a reminder for the user to wear the wearable device.

15. The method of claim 10, further comprising:
generating a baseline heart rate curve associated with the plurality of sleep days based at least in part on the baseline heart rate data; and
generating a heart rate curve associated with the first sleep day based at least in part on the additional heart rate data, wherein identifying the trigger condition is based at least in part on a comparison of the heart rate curve and the baseline heart rate curve.

16. The method of claim 10, further comprising:
generating a baseline heart rate curve based at least in part on a default heart rate curve, physiological data acquired from a plurality of additional users different from the user, or both; and
generating a heart rate curve associated with the first sleep day based at least in part on the additional heart rate data, wherein identifying the trigger condition is based at least in part on a comparison of the heart rate curve and the baseline heart rate curve.

17. The method of claim 10,
wherein the baseline heart rate data and the additional heart rate data comprise heart rates for the user while the user is awake, or
wherein the baseline heart rate data and the additional heart rate data comprise heart rates for the user while the user is asleep.

18. The method of claim 10, further comprising:
identifying the user has awaken from sleep based at least in part on the additional heart rate data, an interaction with the graphical user interface, or both, wherein causing the graphical user interface to display the health-related insight is based at least in part on identifying the user has awaken from sleep.

19. The method of claim 10, further comprising:
determining a bedtime for the user based at least in part on the baseline heart rate data, wherein causing the graphical user interface to display the health-related insight is based at least in part on the bedtime.

20. A method for providing health-related insights to a user, comprising:
receiving baseline physiological data associated with a user via a wearable device, the baseline physiological data collected throughout a plurality of sleep days, wherein the baseline physiological data comprises at least heart rate data and temperature data;
identifying baseline restorative moment data associated with the user based at least in part on the heart rate data and the temperature data, the baseline restorative moment data comprising a first quantity of restorative moments within the plurality of sleep days in which the user is in a relaxed state;
receiving additional physiological data associated with the user via the wearable device, the additional physiological data collected throughout a first sleep day subsequent to the plurality of sleep days;
identifying additional restorative moment data associated with the user based at least in part on the additional physiological data, the additional restorative moment data comprising a second quantity of restorative moments within the first sleep day;
identifying a trigger condition for providing a health-related insight associated with the second quantity of restorative moments based at least in part on a comparison between the additional restorative moment data and the baseline restorative moment data; and
causing a graphical user interface to display the health-related insight associated with the additional physiological data.
